(19) United States
(12) Patent Application Publication
(10) Pub. No.: US 2005/0233313 A1
(43) Pub. Date: Oct. 20, 2005

(54) Methods and reagents for identifying inhibitors of viral protease activity

(76) Inventors: Andrew Kaplan, Chapel Hill, NC (US); Steven Pettit, Durham, NC (US)

Correspondence Address: MYERS BIGEL SIBLEY & SAJOVEC, PO BOX 37428, RALEIGH, NC, 27627, US

(21) Appl. No.: 10/521,381

(22) PCT Filed: Jul. 30, 2003
(86) PCT No.: PCT/US03/23789

§ 371 Date: Jun. 13, 2005

Related U.S. Application Data
(60) Provisional application No. 60/399,462, filed on Jul. 30, 2002.

(51) Int. Cl. C12Q 1/70; C12Q 1/68; C07H 21/02; C12N 15/867

(52) U.S. Cl. 435/5; 435/6; 536/23.72; 435/456

(57) ABSTRACT

The present invention provides reagents and methods for identifying inhibitors of retrovirus protease activity. In particular embodiments, the retrovirus is HIV. Also provided are kits, nucleic acids and proteins for carrying out the methods of the invention.

### RELATED APPLICATION INFORMATION

This application claims the benefit of U.S. Provisional Patent Application No. 60/399,462, filed Jul. 30, 2002, the disclosure of which is incorporated by reference herein in its entirety.

### GOVERNMENT SUPPORT

The present invention was made, in part, with the support of grant number R29 A136702 and GM66681-01 from the National Institutes of Health. The United States government has certain rights to this invention.

### FIELD OF THE INVENTION

The present invention relates to methods and reagents for identifying compounds that inhibit viral activity; in particular, the present invention relates to methods and reagents for identifying inhibitors of viral protease activity.

### BACKGROUND OF THE INVENTION

Activation of the human immunodeficiency virus type 1 (HIV-1) protease (PR) is a critical step in the assembly of HIV-1. The structural and enzymatic proteins that comprise the virus core are initially translated as part of the Gag and GagPol polyprotein precursors. Accurate and ordered processing of these precursors is an essential step in the production of infectious viral particles (Kaplan et al. (1993) J. Virol. 67:4050-4055, Kräusslich et al. (1995) J. Virol. 69:3407-3419, Mervis et al. (1988) J. Virol. 62:3993-4002, Pettit et al. (1994) J. Virol. 68:8017-8027, Wiegers et al. (1998) J. Virol. 72:2846-2854). Processing of the precursors is accomplished by the viral protease, which is also translated as part of the GagPol precursor (Jacks et al. (1988) Nature 331:280-283, Oroszlan and Luftig. (1990) Curr. Top. Microbiol. Immunol. 157:153-185). As is the case for all retroviruses, HIV-1 protease is an aspartic protease and is functional only as a dimer (Loeb et al. (1989) Nature 340:397-400). The active site of the viral protease contains two aspartic acids, each one contributed by monomers that combine to form the dimeric enzyme (Navia et al. (1989) Nature 337:615-620). Roughly half of the interactions that maintain the mature protease dimer occur in a region known as the dimer interface (Weber (1990) J. Biol. Chem. 265:10492-10496). This region is made up of four interdigitating N— and C-terminal residues of the two monomers (residues 1 to 4 and residues 96 to 99) that form a four-stranded β-sheet (Weber (1990) J. Biol. Chem. 265:10492-10496, Wlodawer et al. (1989) Science 245:616-621).

As all of the cleavages within GagPol are accomplished by the viral protease itself, without assistance from a cellular protease, the protease embedded within GagPol must dimerize and be active as part of the GagPol precursor (Oroszlan and Luftig. (1990) Curr. Top. Microbiol. Immunol. 157:153-185). Consequently, the initial cleavages are carried out by the precursor-associated immature protease. This includes the cleavages that release the mature protease itself. Presumably, it is this fully processed, mature protease that is responsible for the later cleavages. Therefore, during virus assembly, the active dimeric enzyme originates as the result of the dimerization of two GagPol precursors. Once the protease domain is liberated from the precursors by cleavage at its N and C termini, a free, mature dimer, consisting of two protease monomers, is produced (Tessmer and Kräusslich (1998) J. Virol. 72:3459-3463).

Despite the wealth of structural data regarding the mature protease dimer, there is little information about the structure of the immature protease dimer that is produced by dimerization of two GagPol precursors. Further, little is known about the changes that accompany the shift from precursor-associated dimer to free enzyme. Finally, the protease must dimerize within the context of precursor dimerization, and several dimerization and oligomerization domains within GagPol have been characterized (Bennett et al. (1993) J. Virol. 67:6487-6498, Bowzard, et al. (1998) J. Virol. 72:9034-9044, Franke et al. (1994) J. Virol. 68:5300-5305, Gamble et al. (1997) Science 278:849-853, Gatlin et al. (1998) J. Biomed. Sci. 5:305-308, Katz and Skalka (1994) Annu. Rev. Biochem. 63:133-173, Quillent et al. (1996) Virology 219:29-36, von Poblotzki et al. (1993) Virology 193:981-985). However, neither the contribution of assembly domains outside the protease to enzyme activation nor the mechanism by which enzyme activation is controlled has been fully assessed.

Thus, there is a need in the art for greater understanding of retrovirus protease activation. Further, there is a need in the art for improved methods of identifying inhibitors of retrovirus protease activity, including inhibitors of the protease activation process.

### SUMMARY OF THE INVENTION

The present invention provides improved methods and targets for identifying inhibitors of retrovirus protease activity. The invention is based, in part, on the discovery that regions outside of the retrovirus protease play a substantial role in protease dimerization and activation and, thus, provides new targets for discovering compounds that inhibit retrovirus protease activity. Using an in vitro system in which the full-length GagPol precursor is expressed and cleaved by the endogenous (i.e., embedded) retrovirus protease, the inventors characterized the initial cleavages of GagPol and the role of the dimer interface in the activation of the protease. The effect of protease dimer interface substitutions on enzyme activity was compared between the mature, free dimer versus the immature, precursor-associated enzyme. Further, interactions within the protease dimer interface that define the specificity of precursor cleavage were characterized. The inventors found that cleavage of the GagPol precursor occurs sequentially and results in the formation of extended protease intermediates. In addition, particular residues of the dimer interface have a role in protease activation as well as the specificity of GagPol cleavages. These studies suggest that assembly domains within GagPol but outside the protease contribute to protease dimerization and activation. Furthermore, these studies indicate that the initial cleavages are intramolecular.

Further, as described in the Examples herein, the inventors have found that known protease inhibitors can be markedly less effective in inhibiting protease activation in vitro within the context of the GagPol precursor as compared with the mature protease. Thus, the retrovirus GagPol (or a fragment thereof including both the protease and regions located outside of the protease) can provide a more robust target for identifying and characterizing inhibitors of retrovirus protease activity. While not wishing to be limited by any particular theory of the invention, it appears that the protease dimer, when embedded within the GagPol precursor, is stabilized by aggregation regions located outside of the protease and is therefore more resistant than the mature protease dimer to compounds that act by disrupting dimer formation. In addition, as discussed above, the inventors have discovered that the initial protease cleavages within the GagPol precursor are intramolecular. The local concentration of the substrate for an intramolecular reaction is much higher than in conventional assays in which a mature protease is added in trans to a substrate (i.e., the cleavage reaction is intermolecular), thereby making the intramolecular reaction more resistant to inhibition. Thus, the screening methods of the invention provide a more stringent system for identifying inhibitors of retrovirus protease activation. In particular, the assay is a more robust system for identifying compounds that bind to the protease active site as well as compounds that act by disrupting the protease dimer.

Accordingly, as one aspect, the present invention provides a method of identifying an inhibitor of retrovirus protease activity, comprising providing a nucleic acid that encodes a retrovirus GagPol or a fragment thereof comprising a protease, a protease cleavage site, a tether and a detectable moiety, wherein either the tether or the detectable moiety is located N-terminal to the cleavage site and the other is located C-terminal to the protease cleavage site; expressing the nucleic acid to produce the retrovirus GagPol or fragment thereof; binding the retrovirus GagPol or fragment thereof to a substrate comprising a binding partner for the tether such that the retrovirus GagPol or fragment thereof is bound via the tether to the substrate; contacting the retrovirus GagPol or fragment thereof with a candidate compound; removing released proteolytic products comprising the detectable moiety; and detecting the level of the detectable moiety bound to the substrate, wherein persistence of the detectable moiety is indicative of an inhibitor of retrovirus protease activity.

As another aspect, the present invention provides a kit for identifying inhibitors of retrovirus protease activity, comprising a nucleic acid that encodes a retrovirus GagPol or a fragment thereof comprising a protease, a protease cleavage site, a tether and a detectable moiety, wherein either the tether or the detectable moiety is located N-terminal to the cleavage site and the other is located C-terminal to the protease cleavage site, such that cleavage at the protease cleavage site results in release of a proteolytic product comprising the detectable moiety; and a substrate comprising a binding partner for the tether.

As a further aspect, the invention provides compounds identified by the methods of the invention.

The present invention also provides nucleic acids that encode a retrovirus GagPol or a fragment thereof comprising a protease, a protease cleavage site, an exogenous tether and an exogenous detectable moiety, wherein either the tether or the detectable moiety is located N-terminal to the protease cleavage site and the other is located C-terminal to the protease cleavage site. Further provided is a retrovirus GagPol or a fragment thereof comprising a protease, a protease cleavage site, an exogenous tether and an exogenous detectable moiety, wherein either the tether or the detectable moiety is located C-terminal to the protease cleavage site and the other is located N-terminal to the protease cleavage site.

These and other aspects of the invention are set forth in more detail in the detailed description of the invention below.

### BRIEF DESCRIPTION OF THE DRAWINGS

FIG. 1. Schematic of the GagPol processing sites and the forced frameshift mutation in pGPfs. (Panel A) Organization of the processing sites in the HIV-1 Gag and GagPol precursor. The Gag and GagPol precursors of HIV-1 are represented as boxes with processing sites as vertical lines. Processed components are given with their accepted nomenclature (Lei et al. (1988) J. Virol. 62:1808-1809). (Panel B) Sequence of the wild-type (WT) HIV in the area of translational frameshift is shown above with the 7-nucleotide heptanucleotide sequence required for translational frameshifting underlined (Reil et al. (1993) J. Virol. 67:5579-5584). The exact site of frameshifting in the virus is variable with 70% of GagPol product containing Leu as the second residue of the TF domain (Gorelick and Henderson (1994) Part III: analyses, p. 2-5. In G. Myers, B. Korber, S. Wain-Hobson, K. T. Jeang, L. Henderson, and G. Pavlakis (ed.), Human retroviruses and AIDS Los Alamos National Laboratory, Los Alamos, N. Mex.). Below is the forced frameshift mutation of pGPfs. pGPfs expresses the major GagPol product in exact sequence. Additional translationally silent substitutions were inserted in the frameshift sequence in pGPfs to improve translational readthrough. The locations of the Gag NC/p1 (Wondrak et al. (1993) FEBS Lett. 333:21-24) and pl/p6 (Henderson et al. (1990) J. Med. Primatol. 19:411-419) sites and the GagPol NC/TF and TF F440/L441 sites are marked with arrows.

FIG. 2. The kinetics of protease activation and the identification of initial cleavages in the GagPol precursor. The full-length GagPol precursor was generated by in vitro transcription and translation in RRL. Aliquots were removed at the indicated time and separated by SDS-PAGE. Wild-type pGPfs is shown on the left. The effect of inhibiting cleavage at the p2/NC, NC/TF, and TF F440/L441 sites with blocking P1 IIe substitutions is shown. The composition and calculated molecular mass of the products based on published sequence are shown on the right. Products are presented in abbreviated form by the N-terminal and C-terminal domains only. Times in minutes (′) and hours (h) are shown above the gels. Kilodaltons are shown on left.

FIG. 3. Effect of single alanine substitutions of the dimer interface on protease activation and specificity within the GagPol precursor. pGPfs (left) or pGPfs containing single alanine substitutions of the eight-dimer interface residues of protease was translated in RRL and separated by SDS-PAGE. Molecular mass markers are shown on the left, and the composition of generated products with estimated molecular mass is shown on the right. Products are abbreviated to the N— and C-terminal domains only. Times in minutes (′) and hours (h) are shown above the gels. Kilodaltons are shown on left. WT, wild type.

FIG. 4. The effect of proline 1 substitutions on the specificity of GagPol cleavage. Proline 1 of the protease domain was replaced by alanine, glycine, phenylalanine, or leucine in the full-length GagPol precursor GPfs prior to translation in RRL and separation by SDS-PAGE. The substitutions resulted in the generation of novel cleavage products (right-facing arrows). Only the P1G substitution inhibited cleavage at the TF/PR, site (loss of 107-kDa intermediate; left-facing arrow). Times in minutes (′) and hours (h) are shown above the gels. Kilodaltons are shown on left. WT, wild type.

FIG. 5. Effect of alanine substitutions of the protease dimer interface residues on the processing of PR-negative GagPol by trans protease. Pr160 produced from the GPfs-PR construct contains a D25A catalytic aspartate substitution of protease that renders the intrinsic protease inactive. Replacement by alanine of the GagPol protease dimer interface residues 1 to 4 and 96 to 99 was evaluated for its effect on processing by wild-type protease provided in trans. Reactions were performed at pH 7, and purified wild-type protease was added at the 0-min time point. Times in minutes (′) and hours (h) are shown above the gels. Kilodaltons are shown on left.

FIG. 6. Effect of multiple-alanine replacement of the residues flanking protease on the activation of the protease within GagPol. (Panel A) An SDS-PAGE gel showing the effect of alanine replacement of the five residues flanking the protease domain on activation of protease and processing of Pr160 GagPol in RRL. Activation and cleavage of the wild-type (WT) GagPol are shown on the left (FS-WT). The P1A substitution results in a active protease with altered specificity of cleavages as shown by the generation of novel products (left arrow). The F99A substitution results in a less active PR. Both the P1A and F99A substitutions enhance cleavage at the TF/PR domain (double arrow). Replacement of the five residues flanking either the N— or C-terminal scissile bond (TF 5A/PR and PR/5A RT mutation) has little effect on either activation or specificity other than preventing cleavage of the mutated processing site. Times in minutes (′) and hours (h) are shown above the gels.

Kilodaltons are shown on left. (Panel B) Schematic of the representative mutations.

FIG. 7. Schematic models of intermolecular (cis) vs. intramolecular (trans) processing for the initial processing of the GagPol precursor. Previous results indicated that initial cleavage of the GagPol precursor by the activated GagPol PR occurs at two sites: p2/NC site (M377/M378) and TF F440/L441 (Pettit et al. (2003) J. Virol. 77:366-374). Cleavage of these GagPol sites could conceivably occur by either an intermolecular mechanism (left) or an intramolecular-mechanism (right). Note that processing of the Gag precursor can only occur by an intermolecular mechanism since the Gag precursor lacks an embedded protease.

FIG. 8A. Initial cleavages of the GagPol precursor in vitro after activation of the protease. Full length GagPol was expressed in vitro as described in Examples 11 and 14. Schematic for the ordered processing of the GagPol precursor after protease activation. Initial cleavage occurred at the p2/NC site (M377/M378) followed by rapid cleavage of the TF F440/L441 site. Significant cleavage of the other GagPol sites was not observed with wild type precursor in vitro. The observed protein products with their calculated molecular mass based on sequence are shown.

FIG. 8B. Initial cleavages of the GagPol precursor in vitro after activation of the protease. Full length GagPol was expressed in vitro as described in Examples 11 and 14. Identification of the location of the initial cleavages and the effect of site-specific blocking mutations on ordered processing of GagPol by the activated protease. The full-length GagPol precursor was generated by in vitro. Aliquots were removed at the indicated time and separated by SDS-PAGE. Processing of the wild type precursor (GP-fs) by the embedded protease is shown. Inactivation of the protease in GagPol with a D25A (GPfs-PR) prevents processing of the Gag precursor. The effect of inhibiting cleavage at the p2/NC or the TF F440/L441 sites with site-specific P1 IIe substitutions is shown. Major products of the GagPol precursor are denoted by dots. The composition and calculated molecular mass of the products based on published sequence are on the right. Products are presented in abbreviated form by their N— and C-terminal domains only according to accepted nomenclature (Leis et al. (1988) J. Virol. 62:1808-1809).

FIG. 9. Comparison of the effect of competitive inhibitor on the cis-activation and trans-processing in vitro. Left: WT GagPol was translated in vitro for 2 hrs in the presence of increasing concentrations of ritonavir (ABT-538) to monitor the effects of the drug on activity of GagPol protease. The protease within GagPol activates and cleaves the precursor at the primary p2/NC and secondary TF F440/L441 sites. The concentration of GagPol in the reaction is approximately 1 nM. The concentration of ritonavir is given above. Right: Effect of ritonavir on trans-cleavage of the GagPol precursor in vitro. 400 nM of mature recombinant protease monomers was added in trans to PR D25A mutated GagPol (60 pM) with varying concentrations of ritonavir (above). Reactions shown were stopped at 20′ incubation. Products are presented in abbreviated form by their N— and C-terminal domains only.

FIG. 10. Plots demonstrating decreased effectiveness of inhibition of the GagPol protease by the competitive inhibitor ritonavir. Plots show the percent inhibition for the individual p2/NC and TF F440/l441 sites for GagPol protease (left) or trans-protease (right) with increasing concentrations of ritonavir. Plots were derived from densitometric analysis of SDS-PAGE gels as described in Example 11. The estimated IC₅₀ for the inhibition of the individual sites for the GagPol protease: TF F440/L441=644 nM; p2/NC=8.25 μM . The estimated IC₅₀for the trans protease: TF F440/L441=18 nM; p2/NC=107nM.

FIG. 11. Trans-complementation test showing that cleavage of the p2/NC and TF F440/L441 sites by the activated GagPol protease occurs by an intramolecular mechanism (cis-restricted) rather than an intermolecular mechanism. GagPol or equal amounts of two GagPol species were expressed in vitro and aliquots were taken at the indicated time prior to SDS-PAGE. A) Wild type GagPol containing protease inactivated at the catalytic aspartate (D25A). B) Wild type GagPol. Panels C-G: trans-complementation test in which equal amounts of two GagPol species were co-translated as shown above the panel. C) Expression of wild type and PR D25A protease. Efficient trans-complementation would be expected to result in 75% inhibition of GagPol precursor processing as shown by the persistence of the full length precursor (75%) and reduced amounts of the 42K MA-p2 product (25%). D, E) Test showing that cleavage of p2/NC site is intramolecular. A mutation that blocks cleavage of the p2/NC site (M377I) was placed on either the GagPol with an active PR monomer (panel D) or the GagPol with an inactive PR monomer (panel E). Processing of the p2/NC site, as shown by the 42 kDa product, is only observed when the unblocked site is located on the same precursor as the active protease, indicating an intramolecular cleavage mechanism (panel E). Panel F, G) Test determining the cleavage mechanism of the TF F440/L441. A mutation that blocks-cleavage of the TF F440/L441 site (F440I) was placed on either the GagPol with an active PR monomer (panel F) or the GagPol with an inactive PR monomer (panel G). Generation of the 113 kDa L441-IN product is seen only when the unblocked site is located on the same precursor as the activated protease, indicating cleavage occurs by a intramolecular mechanism.

FIG. 12. Co-translation test of Gag and GagPol showing that cleavage of the p2/NC site is only cleaved in the GagPol precursor. pGag1 (A) or Gag and GagPol (Panels B-G) were co-translated and aliquots were taken at the indicated times prior to SDS-PAGE. A) Translation of the Gag and Pol open reading frames (pGag1) in RRL resulted in the expression of pr55 Gag and pr160 GagPol by a translational frameshift mechanism (Jacks et al. (1988) Nature 331:280-283). The Gag:GagPol ratio is approximately 20:1. Cleavage of the p2/NC site is evident by the generation of the 42 kDa MA-CA-p2 product B) Co-translation of Gag and GagPol in a 20:1 ratio via separate plasmids. C, D) Test showing that cleavage of p2/NC site is intramolecular. A mutation that blocks cleavage of the p2/NC site (M377I) was placed on either Gag (panel C) or GagPol (panel D) and expressed at a 20:1 ratio. Processing of the p2/NC site, as shown by the 42 kDa product, is only observed when the unblocked site is located GagPol precursor (panel D). Co-expression of Gag and GagPol in a 1:1 ratio. F, G) Gag and GagPol were expressed in a 1:1 ratio in vitro. A mutation that blocks cleavage of the p2/NC site (M377I) was placed on either Gag (panel F) or GagPol (panel G). Cleavage of the p2/NC site, as shown by the 42 kDa MA-CA-p2 product, was only observed when the unblocked site is located GagPol precursor (panel G).

FIG. 13. Schematic of a rapid screen using protease activation to identify inhibitors of protease activity.

FIG. 14. Detectability of p24 GagPol-HA with active and inactive protease by anti-p24 antibody with varying dilutions of GagPol-HA. The x-axis indicates fold-dilution of the indicated GagPol-HA. The concentration of the undiluted stock was 1 μM. Note that maximum signal is given at low dilutions (high concentrations) of GagPol-HA. Readout is given in Luminescence cpm (counts per minute).

FIG. 15. Effect of anti-HA tag antibody dilution on the detection of HA Tag of GagPol-HA with active and inactive protease as captured on anti-p24 antigen plates. The x-axis indicates fold dilution of the anti-HA Tag antibody. Read out is in luminescence cpm (counts per minute). Results indicate that lower dilutions of anti-HA antibody increase the detectablility of HA Tag present. Note that more HA-Tag is detected with an inactive GagPol protease than with an active protease. Background luminescence of 100,000 cpm is subtracted from data.

### DETAILED DESCRIPTION OF THE INVENTION

The present invention is described herein with reference to the accompanying drawings and examples, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount.

All publications, patent applications, patents, and other references mentioned herein are incorporated by reference in their entirety for the teachings described in the sentence and/or paragraph wherein each is mentioned.

Nucleotides and amino acids are represented herein in the manner recommended by the IUPAC-IUB Biochemical Nomenclature Commission, and for amino acids, by either the one-letter code or the three letter code, both in accordance with 37 CFR §1.822 and established usage. See, e.g., Patentln User Manual, 99-102 (November 1990) (U.S. Patent and Trademark Office).

The studies presented in the Examples below examine processing by a retrovirus protease in the context of the full-length Gag and GagPol precursors. Comparisons were made between GagPol processing by the endogenous vs. an exogenous protease and by evaluating processing at more than one cleavage site. This experimental design facilitated identification of differences in the efficiency with which different sites are processed by the endogenous protease and revealed that the context of the protease domain is important for cleavage by the endogenous, but not the exogenously added, protease. As such, the GagPol precursor (or fragment thereof comprising the protease and regions outside of the protease) is a more robust target for identifying inhibitors of protease activation.

In contrast, prior art methods have looked at intermolecular cleavage of a substrate by a mature protease provided in trans and have not appreciated the importance of GagPol regions external to the protease on protease activity. Thus, the inventors have found that an inhibitor that is effective against a mature protease can be much less effective in inhibiting the protease in the context of the GagPol precursor.

Accordingly, the present invention provides methods for identifying an inhibitor of virus protease activity. In one representative embodiment of the invention, the method for identifying an inhibitor of virus protease activity comprises providing a nucleic acid that encodes a retrovirus GagPol or a fragment thereof comprising a protease, a protease cleavage site, a tether and a detectable moiety, wherein either the tether or the detectable moiety is located N-terminal to the cleavage site and the other is located C-terminal to the protease cleavage site; expressing the nucleic acid to produce the retrovirus GagPol or a fragment thereof; binding the a retrovirus GagPol or fragment thereof to a substrate comprising a binding partner for the tether such that the a retrovirus GagPol or fragment thereof is bound via the tether to the substrate; contacting the retrovirus fragment with a candidate compound; removing released proteolytic products comprising the detectable moiety; and detecting the level of the detectable moiety bound to the substrate, wherein persistence of the detectable moiety is indicative of an inhibitor of retrovirus protease activity.

The methods carried out according to this embodiment of the invention are suitable for use as high throughput discovery protocols. The full-length GagPol or fragment thereof comprising the protease and a protease cleavage site is bound or “tethered” to a solid substrate. Activation of the protease results in cleavage at the protease cleavage site within the GagPol or fragment thereof. There is a detectable moiety (e.g., a label or an epitope) on the other side of the cleavage site from the tether, and cleavage at the protease site will release a proteolytic product(s) comprising the detectable moiety from the substrate. Thus, protease activity can be monitored by loss of the detectable moiety from the substrate. If an inhibitor of protease activity is added to the system, there will be a diminishment or lessening in the loss of the detectable moiety from the substrate (i.e., there will be a persistence in the detectable moiety).

The present invention can be carried out using any virus protease (or protease embedded within a precursor) known in the art. In this embodiment, the target is a precursor comprising the virus protease, a virus cleavage site, a tether and a detectable moiety, wherein either the tether or the detectable moiety is located C-terminal to the cleavage site and the other is located N-terminal to the protease cleavage site. Exemplary virus proteases include, but are not limited to, proteases from the Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt;: Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; lridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea venation mosaic virus group; Phycodnaviridae; Picomaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites. See FIELDS et al., VIROLOGY, volume 2, chapter 67 (3d ed., Lippincott-Raven Publishers, for discussion of these and other viruses.

In particular-embodiments, the virus protease is from a retrovirus, a rhinovirus, a poxvirus, a hepatitis C virus, a Yellow fever virus, a poliovirus or a smallpox virus.

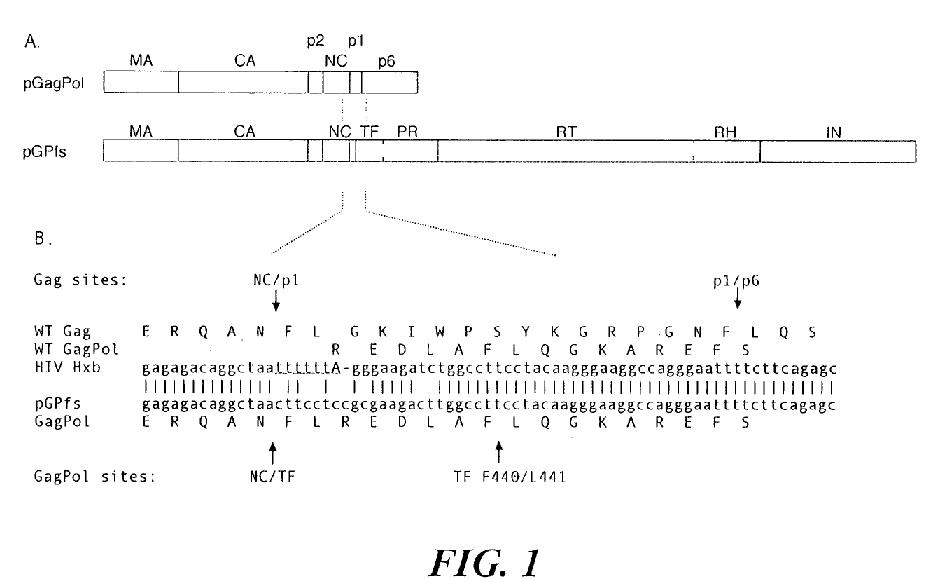

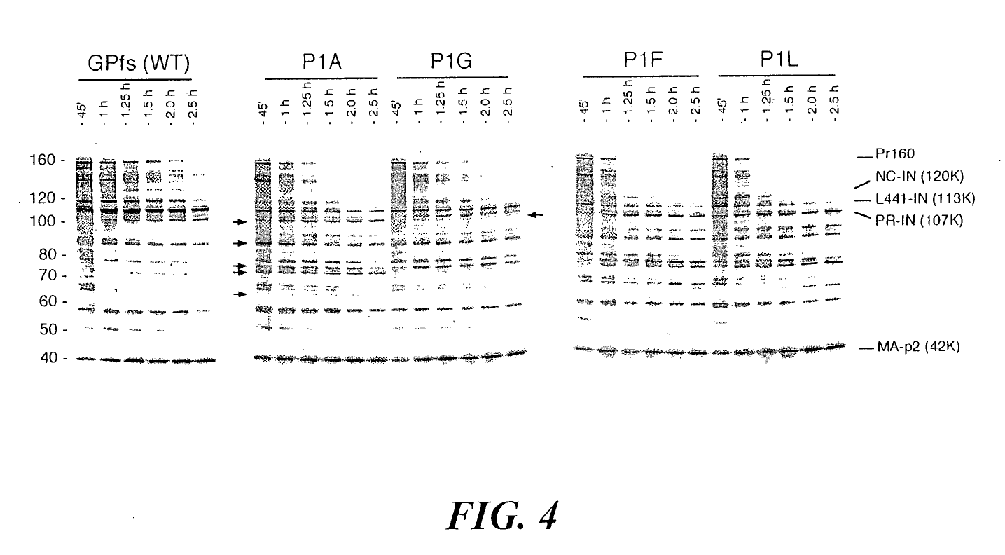

In other representative embodiments, the virus protease is a retrovirus protease. The genomic structure of retroviruses is well understood in the art (see, e.g., FIELDS et al., VIROLOGY, volume 2, chapters 57-62 (3d ed., Lippincott-Raven Publishers), in particular, Chapter 59, FIG. 4; see also FIG. 1 herein). In particular, the organization of the GagPol precursor is well conserved across different retroviruses and has been extensively characterized. Retrovirus proteases include but are not limited to proteases from the Alpharetrovirus genus (e.g., Avian leucosis virus and Rous sarcoma virus), Betaretrovirus genus (e.g., Mouse mammary tumor virus, Mason-Pfizer monkey virus, Jaagsiekte sheep retrovirus), Gammaretrovirus genus (e.g., Murine leukemia viruses, Feline leukemia virus, Gibbon ape leukemia virus, reticuloendotheliosis virus), Deltaretrovirus genus (e.g., Human T-lymphotrophic virus, Bovine leukemia virus, Simian T-lymphotrophic virus), Epsilonretrovirus (e.g., Walleye dermal sarcoma virus, walleye epidermal hyperplasia virus 1), lentivirus genus (e.g., Human immunodeficiency virus [HIV], including HIV-1 and HIV-2, Simian immunodeficiency virus, Equine infectious anemia virus, Feline immunodeficiency virus, Caprine arthritis encephalitis virus, Visna/maedi virus) and the Spumavirus genus (e.g., Human foamy virus). In particular embodiments, the retrovirus protease is derived from a lentivirus, more particularly from HIV.

In other particular embodiments, the protease is from a “resistant” retrovirus strain. The term “resistant” retrovirus strain has its conventional meaning in the art, e.g., a strain that has acquired mutations that render it less susceptible to therapeutic agents or therapies that are partially or completely effective against the wild-type virus. Numerous resistant retrovirus strains are known in the art. For example, there are a number of publicly-available websites listing resistant HIV strains (see, e.g., the databases of the International AIDS Society-USA at http://www.iasusa.org, and Los Alamos National Laboratory at http://resdb.lanl.gov/Resist_DB/default.htm).

In illustrative embodiments of the invention, the protease is embedded within a full-length retrovirus GagPol precursor. The nucleic acid and amino acid sequences of numerous GagPol precursors are known in the art. For example, the nucleic acid and amino acid sequence of the complete genome of HIV-1, including the GagPol precursor, has been deposited under GenBank Accession No. NC_{—}001802.

Alternatively, in other embodiments of the present invention, the nucleic acid encodes a retrovirus GagPol fragment comprising a protease. The fragment can comprise regions outside of the protease region (e.g., extending in the N-terminal direction into the transframe region and/or C-terminal direction into the reverse transcriptase region). In particular embodiments, the fragment comprises from about 10, 20, 50, 75, 100, 150, 200, 300 amino acids or more from the GagPol precursor in the N-terminal and/or C-terminal direction from the protease.

The fragment can comprise any of the elements of a GagPol precursor in any combination. These elements include, but are not limited to the retrovirus protease, the retrovirus transframe protein, the retrovirus p1 protein, the retrovirus nucleocapsid protein, the retrovirus p2 protein, the retrovirus capsid protein, the retrovirus matrix protein, the retrovirus reverse transcriptase, the retrovirus RNase H, and the retrovirus integrase.

Turning to FIG. 1, in particular embodiments, the fragment comprises, consists essentially of, or consists of the retrovirus protease and all, or a portion of, the transframe protein. The fragment can further comprise, consist essentially of, or consist of all, or a portion of, the p1 protein. The fragment can further comprise, consist essentially of, or consist of all, or a portion of, the nucleocapsid protein. The fragment can still further comprise, consist essentially of, or consist of all, or a portion of, the p2 protein. The fragment can further comprise, consist essentially of, or consist of all, or a portion of, the capsid protein. Still further, the fragment can comprise, consist essentially of, or consist of all, or a portion of, the matrix protein.

Alternatively, or additionally, the fragment can extend in the C-terminal direction to comprise, consist essentially of, or consist of the protease and all, or a portion of, the reverse transcriptase. The fragment can further comprise, consist essentially of, or consist of all, or a portion of the RNase H protein. The fragment can still further comprise, consist essentially of, or consist of all, or a portion of, the integrase.

In other particular embodiments, the GagPol fragment comprises the cleavage site within the transframe protein (e.g., at F440/L441 of HIV-1; see GenBank Accession No. NC 001802) and/or the cleavage site between p2/nucleocapsid.

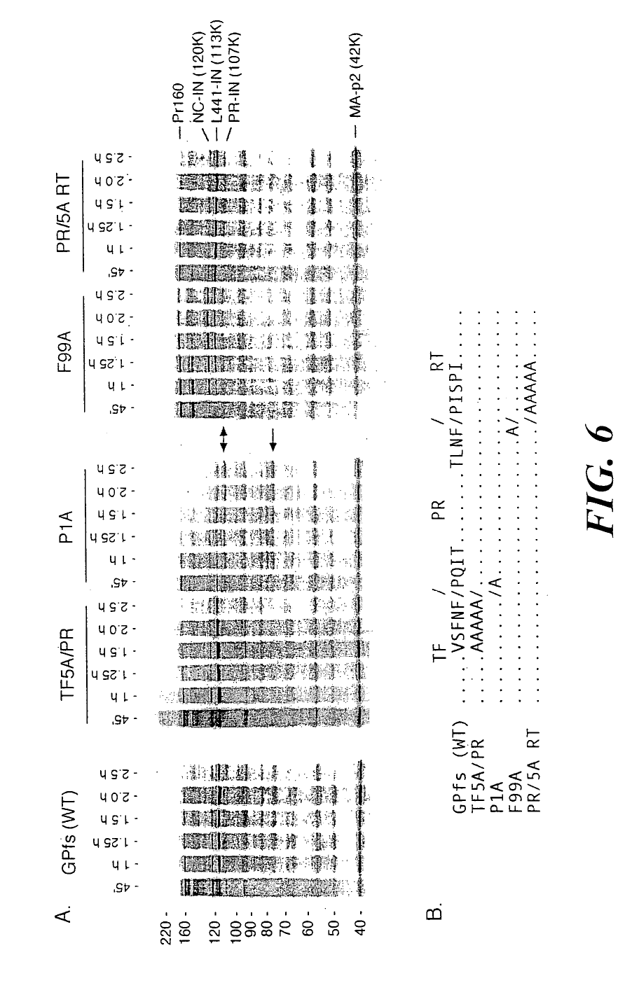

The GagPol precursor further contains one or more protease cleavage sites. For ease and convenience, the protease cleavage site will generally be an endogenous site found within the GagPol precursor or fragment thereof. However, those skilled in the art will appreciate that a synthetic protease cleavage site(s) can be introduced into (including insertion at the ends of) the GagPol precursor or fragment thereof. The protease cleavage sites of many retrovirus proteases are known in the art. To illustrate, some of the amino acid sequences of the HIV-1 protease cleavage sites are presented in FIG. 6 herein. There is variability observed in the protease cleavage sites, and other HIV-1 protease cleavage sites can be found in publicly available databases maintained by the Los Alamos National Laboratory at http://resdb.lanl.gov/Resist DB/default.htm. Further, as demonstrated in the Examples below, the GagPol precursor or fragment can be modified to ablate one or more of the endogenous protease cleavage sites, e.g., to study inhibition of protease activity at a particular cleavage site of interest, by introducing a frameshift, substitution, deletion, insertion and the like into one or more of the protease cleavage sites.

In one representative embodiment, the GagPol fragment comprises, consists essentially of, or consists of the protease with a 4 amino acid extension on each of the N— and C-terminal ends to reconstitute the protease cleavage sites. Likewise, the protease cleavage sites can be recreated by extensions of other GagPol fragments (e.g., the cleavage site at the N-terminal end of the nucleocapsid protein).

By “retrovirus protease activity” it is meant the level, degree, extent, speed and/or efficiency of protease cleavage at the cleavage site(s) in the GagPol precursor or fragment thereof. Those skilled in the art will appreciate that the methods of the invention are not to be limited by the mechanism of cleavage. For example, the inventors have found that the initial protease cleavages are intramolecular. However, in particular embodiments the invention also, or alternatively, encompasses methods in which intermolecular cleavage occurs.

The term “nucleic acid” is intended to refer to a nucleic acid molecule (e.g., DNA or RNA or a chimera thereof). The nucleic acid encoding the GagPol precursor or fragment thereof can be derived from a natural source or, alternatively, can be partially or entirely synthetic. As described in the Examples, in particular embodiments, the retrovirus coding sequences for the GagPol precursor are modified to resolve the frameshift of the Pol reading frame as compared with the Gag reading frame, thereby enhancing readthrough the Pol coding sequences.

The nucleic acid can be provided by a vector (typically, an expression vector). Any suitable vector known in the art can be used to provide the nucleic acid encoding the GagPol precursor or fragment thereof. Exemplary vectors include but are not limited to plasmids, BACs, YACs, phage, cosmids and viral vectors (e.g., adenovirus, EBV, AAV, baculovirus, herpesvirus, and the like).

Standard techniques for the construction of the vectors of the present invention are well-known to those of ordinary skill in the art and can be found in such references as Sambrook et al. (1989) Molecular Cloning: A Laboratory Manual 2nd Ed. Cold Spring Harbor, N.Y. and F. M. Ausubel et el. (1994) Current Protocols in Molecular Biology Green Publishing Associates, Inc. and John Wiley & Sons, Inc., New York, N.Y. A variety of strategies are available for ligating fragments of DNA, the choice of which depends on the nature of the termini of the DNA fragments and which choices can be readily made by the skilled artisan.

As used herein, an “isolated” nucleic acid or polypeptide means a nucleic acid or polypeptide separated or substantially free from at least some of the other components of the naturally occurring organism or virus, for example, the cell or viral structural components or other polypeptides or nucleic acids commonly found associated with the nucleic acid or polypeptide.

As used herein, the term “polypeptide” encompasses both peptides and proteins, unless indicated otherwise.

As noted above, the nucleic acid encodes a retrovirus GagPol or fragment thereof comprising a protease and further comprising a “tether.” The terms “tether”, “tethered”, “tethering” and the like as used herein refers to a portion of the GagPol precursor or fragment thereof (e.g., an epitope) or a physical modification of the protein (e.g., the addition of a ligand or “tag”) that will interact with a binding partner attached to a substrate so as to adhere or bind the GagPol precursor or fragment to the substrate. The tether can be 3, 4, 5, 7, 10, 15, 20, 25, 30, 50, 70, 99, 100, 200 or any number of amino acids long. Further, the tether can be artificially or naturally constructed. Methods of adhering polypeptides to solid substrates are well known in the art, and any suitable mention can be used in connection with the present invention.

In particular embodiments, the tether may be an epitope of the retrovirus GagPol or a fragment thereof. For example, the tether can be an epitope within the capsid, matrix, nucleocapsid, p1, p2, p6, transframe, protease, reverse transcriptase, RNase H or integrase proteins, and the binding partner can be an antibody that specifically binds to the epitope. In representative embodiments, the tether is an epitope within the retrovirus capsid protein.

Alternatively, the tether can be an exogenous (i.e., foreign to the GagPol) epitope or chemical tag that is inserted into or at the N-terminal or C-terminal end of the full-length GagPol precursor or fragment. For convenience, the chemical tag can be a polypeptide tag. Alternatively, chemical modifications can be introduced into the GagPol precursor or fragment thereof that function as a tag (e.g., the N-terminal amino acid can be modified using known chemical methods). In particular embodiments, the tether can be, without limitation, an exogenous epitope, an enzyme, a ligand, a receptor, an antibody or antibody fragment, biotin and the like. In other representative embodiments, the tether is a hemagglutinin antigen, Protein A, polyHis (e.g., for binding to Ni), maltose binding protein, c-myc, FLAG epitope, glutathione-S-transferase, horseradish peroxidase, alkaline phosphatase, or strepavidin.

The tether is recognized by a binding partner that is bound or affixed to the substrate. The choice of binding partner depends on the particular tether and may be, without limitation, an antibody or antibody fragment, nickel, a polypeptide, a receptor, a ligand, a nucleic acid, a polysaccharide, biotin and the like. Methods of binding or affixing binding reagents to a wide array of solid substrates are known in the art.

The tether and binding partner form a “binding pair,” which refers to a pair of molecules that specifically and selectively bind to one another. Examples of suitable binding pairs include, but are not limited to: nucleic acid and nucleic acid; protein or peptide and nucleic acid; protein or peptide and protein or peptide; antigens and antibodies; receptors and ligands, haptens, or polysaccharides, etc. Members of binding pairs are sometimes also referred to as “binders” herein.

The nucleic acid further encodes a detectable moiety, which can be any label or chemical tag inserted into or attached at the N— or C-terminal end of the full-length GagPol precursor or fragment. The detectable moiety can be naturally or artificially constructed. Reagents and methods for detecting polypeptides are well-known in the art, and any suitable method can be used with the present invention.

In particular embodiments, the detectable moiety is a portion (e.g., an epitope) of the retrovirus GagPol or a fragment thereof. For example, the detectable moiety can be an epitope within the capsid, matrix, nucleocapsid, p1, p2, p6, transframe, protease, reverse transcriptase, RNase H or integrase protein.

Alternatively, the detectable moiety can be an exogenous epitope or chemical tag that is inserted into or at the N-terminal or C-terminal end of the full-length GagPol precursor or fragment. The detectable moiety can be any exogenous label or tag that can be detected using any method known in the art. According to this embodiment, the detectable moiety can be an epitope, an enzyme, a ligand, a receptor, an antibody or antibody fragment and the like. In representative embodiments, the tether is a hemagglutinin antigen, polyHis, biotin, Protein A, strepavidin, maltose binding protein, c-myc, FLAG epitope, glutathione-S-transferase, alkaline phosphatase, horseradish peroxidase, a fluorescent moiety (e.g., Green Fluorescent protein), β-glucuronidase, β-galactosidase, luciferase or a radioisotope.

The detectable moiety can be detected either directly or indirectly. For example, for direct detection, the GagPol or fragment can be tagged with a radioisotope (e.g., ³⁵S) and the presence of the radioisotope detected by autoradiography. As another example, the GagPol or fragment can be tagged with a fluorescent moiety and detected by fluorescence as is known in the art. Alternatively, the detectable moiety can be indirectly detected by tagging the GagPol precursor or fragment thereof with a detectable moiety that requires additional reagents to render it detectable. Illustrative methods of indirect labeling include those utilizing chemiluminescence agents, chromogenic agents, enzymes that produce visible reaction products, and ligands (e.g., haptens, antibodies or antigens) that may be detected by binding to labeled specific binding partners (e.g., hapten binding to a labeled antibody).

In particular embodiments, the detectable moiety is an antibody or antibody fragment. A variety of protocols for detecting the presence of and/or measuring the amount of polypeptides, using either polyclonal or monoclonal antibodies or fragments thereof are known in the art. Examples of such protocols include, but are not limited to, enzyme-linked immunosorbent assays (ELISA), radioimmunoassays (RIA), radioreceptor assay (RRA), immunoprecipitation, Western blotting, competitive binding assays and immunofluorescence. These and other assays are described, among other places, in Hampton et al. (Serological Methods, a Laboratory Manual, APS Press, St Paul, Minn. (1990)) and Maddox et al. (J. Exp. Med. 158:1211-1216 (1993)). In a representative embodiment of this aspect of the invention, the substrate is a microtiter plate and the method comprises an ELISA.

Those skilled in the art will appreciate that the detectable moiety and tether are typically selected so that they are different molecules. Further, the tether and detectable moiety are generally chosen so that there is no significant cross-reactivity therebetween, i.e., the detectable label will not bind significantly to the binding partner on the substrate and the tether will not interact to a significant extent with reagents for detecting the detectable moiety. The tether can be located N-terminal or C-terminal to a protease cleavage site in the GagPol precursor or fragment thereof. Likewise, the detectable moiety can be located N-terminal or C-terminal to the protease cleavage site. Generally, the tether and detectable moiety are on opposite sides of a protease cleavage site, so that upon cleavage, the tether remains bound to the substrate, but the proteolytic product comprising the detectable moiety is released from the substrate. The position of the protease within the GagPol precursor or fragment thereof is not critical, e.g., the tether and detectable moiety can be oriented on either side of the protease, alternatively, these elements can both be positioned N-terminal or C-terminal to the protease.

In particular representative embodiments of the present invention, the nucleic acid encodes a full-length GagPol or a fragment thereof comprising at least the protease, transframe, p1., nucleocapsid, p2 and capsid proteins. The protease cleavage site(s) is a native cleavage site within the GagPol or precursor. An epitope within the capsid protein is the tether and an antibody directed against the capsid (e.g., anti-p24 antibody) is the binding partner that will tether the GagPol precursor or GagPol fragment to the substrate. At the C-terminal end of the molecule, a detectable moiety (e.g., influenza HA) is fused to the GagPol or precursor thereof.

In embodiments wherein the tether and/or the detectable moiety are polypeptides that are exogenous to the GagPol precursor or fragment thereof, the GagPol precursor or fragment is a fusion polypeptide that comprises two or more polypeptides covalently linked together, e.g., by peptide bonding. Likewise, according to this embodiment, the nucleic acid encoding the GagPol precursor or fragment comprises two or, more nucleic acid sequences covalently linked together by methods standard in the art.

The nucleic acid is “expressed” to produce the GagPol or fragment thereof comprising the protease, protease cleavage site, tether and detectable moiety. By “express,” “expresses,” “expression,” “expressed” and grammatical variations thereof it is meant transcription and translation to produce the GagPol precursor or fragment thereof. Methods for in vitro transcription and translation of nucleic acids are well-known in the art. For example, rabbit reticulocyte lysate (RRL) systems are commercially available and widely used for this purpose.

The GagPol precursor or fragment can be expressed from the nucleic acid in the presence of the substrate (e.g., in an ELISA plate). Alternatively, the nucleic acid can be expressed to produce the GagPol or fragment thereof, which can then be contacted with and bound to the substrate (e.g., by adding beads for immunopurification or by putting the translation reaction products over a chromatography column). The GagPol or fragment is adhered or bound to the substrate via interaction between the tether and its binding partner affixed to the substrate.

The bound GagPol or fragment thereof is contacted with a candidate compound to be tested for inhibition of retrovirus protease activity and/or a known retrovirus protease inhibitor. Any candidate compound known in the art can be used in the methods of the invention. In particular, modified versions of known retrovirus protease inhibitors can be tested. Known protease inhibitors include but are not limited to amprenavir, atazanavir, indinavir, lopinavir, nelfinavir, ritonavir and saquinavir. Other retrovirus inhibitors that are suitable for the methods of the invention include inhibitors of virus capsid assembly that act by disrupting Gag or GagPol association. See also, Yao et al., (1998) Endothiopeptide inhibitors of HIV-1 protease. Bioorganic and Medicinal Chemistry Letters 8:699-704 (describing protease inhibitors that prevent protease dimerization).

Any compound of interest can be screened for inhibition of retrovirus protease activity according to the present invention. Suitable test compounds include small organic compounds (i.e., non-oligomers), oligomers or combinations thereof, and inorganic molecules. Suitable organic molecules can include but are not limited to polypeptides (including enzymes, antibodies and Fab′ fragments), carbohydrates, lipids, coenzymes, and nucleic acid molecules (including DNA, RNA and chimerics and analogs thereof) and nucleotides and nucleotide analogs. In particular embodiments, the compound is an antisense nucleic acid, an siRNA or a ribozyme that inhibits production of the GagPol or fragment thereof.

Small organic compounds (or “non-oligomers”) include a wide variety of organic molecules, such as heterocyclics, aromatics, alicyclics, aliphatics and combinations thereof, comprising steroids, antibiotics, enzyme inhibitors, ligands, hormones, drugs, alkaloids, opioids, terpenes, porphyrins, toxins, catalysts, as well as combinations thereof.

Oligomers include oligopeptides, oligonucleotides, oligosaccharides, polylipids, polyesters, polyamides, polyurethanes, polyureas, polyethers, and poly (phosphorus derivatives), e.g. phosphates, phosphonates, phosphoramides, phosphonamides, phosphites, phosphinamides, etc., poly (sulfur derivatives) e.g., sulfones, sulfonates, sulfites, sulfonamides, sulfenamides, etc., where for the phosphorous and sulfur derivatives the indicated heteroatom for the most part will be bonded to C,H,N,O or S, and combinations thereof. Such oligomers may be obtained from combinatorial libraries in accordance with known techniques.

Further, the methods of the invention can be practiced to screen a compound library, e.g., a combinatorial chemical compound library (e.g., benzodiazepine libraries as described in U.S. Pat. No. 5,288,514; phosphonate ester libraries as described in U.S. Pat. No. 5,420,328, pyrrolidine libraries as described in U.S. Pat. Nos. 5,525,735 and 5,525,734, and diketopiperazine and diketomorpholine libraries as described in U.S. Pat. No. 5,817,751), a polypeptide library, a cDNA library, a library of antisense nucleic acids, and the like, or an arrayed collection of compounds such as polypeptide and nucleic acid arrays.

The contacting step is generally carried out in a liquid solution (e.g., an aqueous solution). Any suitable concentration of the candidate compound can be added to the assay. Typically, the concentration of the candidate compound can range from 0.1 nM to 100 mM. In particular embodiments, two or more concentrations of the compound are used so that an IC₅₀ (concentration at which 50% inhibition is achieved) can be determined.

In embodiments of the invention, the candidate compound(s) can be added to the bound GagPol precursor or fragment (i.e., after translation thereof from the nucleic acid). Alternatively, it can be advantageous to have the candidate compound already present as the GagPol precursor or fragment is translated so as to prevent significant levels of protease activation occurring prior to addition of the candidate compound.

The methods of the invention are carried out to identify compounds that inhibit retrovirus protease activity. Thus, compounds identified according to these methods are also an aspect of the invention.

By “inhibition” of retrovirus protease activity, it is meant that the candidate compound reduces or diminishes the level, degree, extent, speed and/or efficiency of protease cleavage at the protease cleavage site(s).

Those skilled in the art will appreciate that inhibition need not be complete, but may be partial. In particular embodiments, at least about a 20%, 25%, 35%, 50%, 65%, 75%, 85%, 90%, 95%, 97%, 98%, 99% or more inhibition is observed as compared with the level of protease activity in the absence of the candidate compound. As an alternate statement, in illustrative embodiments, an inhibitory compound according to the present invention has an IC₅₀ that is less than about 1 mM, 500 μM, 10.0 μM, 50 μM, 1 μM or less.

Detecting inhibition of protease activity using the methods of the present invention is independent of the mechanism of inhibition, and thus the invention is not to be limited by any particular mechanism of inhibition. To illustrate, inhibition of protease activity can be achieved by inhibiting protease activation. As further non-limiting examples, the compound can inhibit protease activity by binding to a cleavage site, preventing dimer formation, inhibiting catalytic activity of the protease and the like. Potential targets for the inhibitory compound are not limited to the protease itself, but extend to other regions of the GagPol precursor (e.g., matrix, capsid, p2, p1, p6, transframe, reverse transcriptase, RNase H and/or integrase regions).

Protease activity results in cleavage at the cleavage site and release of a proteolytic product(s) comprising the detectable moiety. Thus, protease activity can be evaluated by following or measuring the decrease in bound levels of the detectable moiety. Typically, the released proteolytic products are removed (e.g., by washing) from the cleaved and uncleaved polypeptides bound to the substrate prior to detecting the level of detectable moiety still bound to the substrate. Alternatively, the protease activity can be monitored by measuring the level of detectable moiety released from the plate (e.g., in the wash solution).

Particular embodiments of the present invention include detecting the level of the detectable moiety bound to the substrate, wherein persistence of the detectable moiety is indicative of an inhibitor of retrovirus protease activity. By the term “persistence” or “persistent” it is intended that release of the detectable moiety from the substrate is lessened or reduced as compared with the level observed in the absence of the candidate compound(s). These terms are not intended to mean that inhibition of the protease is complete and there is no reduction in the detectable moiety bound to the substrate. As an alternative statement, “persistence” of the detectable moiety indicates that less than about 50%, 35%, 25%, 15%, 10%, 5%, 2%,1 % or less of the detectable moiety is released from the substrate in the presence of the candidate compound.

Additional embodiments include detecting the level of the detectable moiety bound to the substrate, wherein a reduction in the level of the detectable moiety bound to the substrate is indicative of protease activation, thereby identifying an inhibitor of GagPol or a fragment thereof protease activity.

The persistence of the detectable moiety bound to the substrate and the inhibition of protease activity can be defined with reference to a predetermined standard. For example, the reduction in the detectable moiety can be lessened by about 25%, 35%, 50%, 65%, 75%, 85%, 90%, 95%, 98%, 99% or more in the presence of the candidate compound as compared with the predetermined standard. Alternatively, the predetermined standard defines a threshold value for inhibitory activity.

The predetermined standard can be a fixed value (e.g., at least 50% inhibition) or can be defined relative to the particular assay (e;g., as compared with no-compound controls). As a further alternative, the predetermined standard can be fixed with reference to a known protease inhibitor (as described above).

Use of a predetermined standard-with the present invention can be particularly advantageous in a high throughput format in that a predetermined threshold level of inhibition can be set and only those compounds that reach or surpass that threshold are identified for further study and characterization.

The screening methods of the invention can be qualitative or quantitative. Quantitative methods can be used to more fully characterize the inhibitory activity of the candidate compound. For example, persistence of, or reductions in, the level of the detectable moiety can be expressed as an IC₅₀ value for the candidate compound. Inhibition of protease activity can also be quantified by standard kinetic assays. See, Ferhst, A. (1977), Enzyme Structure and Mechanisms, W. H. Freeman and Co., San Francisco; Segal, I. H. (1975), Enzyme Kinetics, John Wiley and Sons, New York).

A number of solid substrates are available to the skilled artisan for use according to the present invention. Solid phases useful as substrates for the present invention include but are not limited to polystyrene, polyethylene, polypropylene, polycarbonate, or any solid plastic material in the shape of test tubes, beads, microparticles, dip-sticks, plates, and the like. Additional substrates include, but are not limited to membranes, multi-well plates (e.g., 96-well microtiter plates), test tubes and Eppendorf tubes. Substrates also include glass beads, glass test tubes and any other appropriate glass vessel. A functionalized solid phase such as plastic or glass, which has been modified so that the surface carries carboxyl, amino, hydrazide, or aldehydes groups can also be used. In general such substrates comprise any surface wherein a binding agent can be attached or a surface which itself provides a binding site. Alternatively, the substrate can be a bead (e.g., for immunoprecipitation) a filter paper, or a chromatography matrix (e.g., anion-exchange, cation-exchange, or affinity) comprising the binding partner.

The methods of the invention can be completely manual, or alternatively, they can be partially or completely automated. Methods to evaluate a large number of samples (e.g., greater than about 50, 100, 200, 300, 500, 800, 1000, 2000, 5000 samples or more) will generally be at least partially automated to facilitate high throughput of samples. For example; the data can be captured and analyzed using an automated system.

As a further aspect, the present invention provides kits for identifying inhibitors of retrovirus protease activity, comprising a nucleic acid that encodes a retrovirus GagPol or a fragment thereof comprising a protease, a protease cleavage site, a tether and a detectable moiety, wherein either the tether or the detectable moiety is located N-terminal to the cleavage site and the other is located C-terminal to the protease cleavage site, such that cleavage at the protease cleavage site results in release of a proteolytic product comprising the detectable moiety; and a substrate comprising a binding partner for the tether (all these terms are as defined hereinabove). Additionally, the kit can further comprise reagents for expressing the nucleic acid e.g., a rabbit reticulocyte lysate [RRL]. In other illustrative embodiments, the kit comprises reagents for detecting the detectable moiety (e.g., a radiolabel, enzyme, enzyme substrates, antibodies, and the like). The kit can further comprise other reagents such as enzymes (e.g., RNA polymerase), salts, buffers, detergents and the like for carrying out the inventive method. The components of the kit are packaged together in a common container, typically including instructions for performing selected specific embodiments of the methods disclosed herein.

The present invention also encompasses a nucleic acid encoding a retrovirus GagPol or a fragment thereof comprising a protease, a protease cleavage site, an exogenous tether and an exogenous detectable moiety, wherein either the tether or the detectable moiety is located N-terminal to the protease cleavage site and the other is located C-terminal to the protease cleavage site. By “exogenous” it is meant that the tether and detectable label are not portions (e.g., epitopes) within the GagPol or fragment itself. As described above, the nucleic acid can be provided by a vector comprising the nucleic acid. Additionally, as a further embodiment, the present invention provides a retrovirus GagPol precursor or fragment thereof comprising a protease, a protease cleavage site, an exogenous tether and an exogenous detectable moiety, wherein one of the tether and the detectable moiety is located N-terminal to the cleavage site and the other is located C-terminal to the protease cleavage site.

In general, any retrovirus protease, GagPol precursor, or fragment thereof known in the art can be used according to the present invention. In particular embodiments, the nucleic acid encoding the retrovirus protease, GagPol precursor or fragment thereof has at least about 50%, 60%, 70%, 75%, 80%, 85%, 90%, 95%, 98% or more nucleic acid sequence homology with the sequences specifically disclosed herein. The term “homology” as used herein refers to a degree of complementarity between two or more sequences. There can be partial homology or complete homology (i.e., identity). A partially complementary sequence that at least partially inhibits an identical sequence from hybridizing to a target nucleic acid is referred to using the functional term “substantially homologous.” The inhibition of hybridization of the completely complementary sequence to the target sequence can be examined using a hybridization assay (Southern or northern blot, solution hybridization and the like) under conditions of low stringency. A substantially homologous sequence or hybridization probe will compete for and inhibit the binding of a completely homologous sequence to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding can be tested by the use of a second target sequence, which lacks even a partial degree of complementarity (e.g., less than about 30% identity). In the absence of non-specific binding, the probe will not hybridize to the second non-complementary target sequence.

Alternatively stated, in particular embodiments, nucleic acids encoding a protease, GagPol precursor, or fragment thereof that hybridize to the complement of the sequences specifically disclosed herein can also be used according to the present invention. The term “hybridization” as used herein refers to any process by which a first strand of nucleic acid binds with a second strand of nucleic acid through base pairing.

The term “stringent” as used here refers to hybridization conditions that are commonly understood in the art to define the commodities of the hybridization procedure.

High stringency hybridization conditions that will permit homologous nucleotide sequences to hybridize to a nucleotide sequence as given herein are well known in the art. For example, hybridization of such sequences to the nucleic acid molecules disclosed herein can be carried out in 25% formamide, 5×SSC, 5× Denhardt's solution, with 100 g/ml of single-stranded DNA and 5% dextran sulfate at 42 C, with wash conditions of 25% formamide, 5×SSC, 0.1% SDS at 42 C for 15 minutes, to allow hybridization of sequences of about 60% homology. More stringent conditions are represented by a wash stringency of 0.3 M NaCl, 0.03 M sodium citrate, 0.1% SDS at 60 C or even 70 C using a standard hybridization assay (see SAMBROOK et al., EDS., MOLECULAR CLONING: A LABORATORY MANUAL 2d ed. (Cold Spring Harbor, N.Y. 1989).

In other embodiments, the amino acid sequence of the protease, GagPol precursor or fragment thereof has at least about 50%, 60%, 70%, 75%, 80%, 85%, 90%, 95%, 98% or more sequence homology with the amino acid sequences disclosed herein.

As is known in the art, a number of different programs can be used to identify whether a nucleic acid or amino acid has sequence identity or similarity to a known sequence. Sequence identity or similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48, 443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp, CABIOS 5,151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/ README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity. An additional useful algorithm is gapped BLAST as reported by Altschul et al. Nucleic Acids Res. 25, 3389-3402.

The CLUSTAL program can also be used to determine sequence similarity. This algorithm is described by Higgins et al. (1988) Gene 73:237; Higgins et al. (1989) CABIOS 5:151-153; Corpet et al. (1988) Nucleic Acids Res. 16: 10881-90; Huang et al. (1992) CABIOS 8: 155-65; and Pearson et al. (1994) Meth. Mol. Biol. 24: 307-331.

The alignment may include the introduction of gaps in the sequences to be aligned. In addition, for sequences which contain either more or fewer nucleotides than the nucleic acids disclosed herein, it is understood that in one embodiment, the percentage of sequence identity will be determined based on the number of identical nucleotides acids in relation to the total number of nucleotide bases. Thus, for example, sequence identity of sequences shorter than a sequence specifically disclosed herein, will be determined using the number of nucleotide bases in the shorter sequence, in one embodiment. In percent identity calculations relative weight is not assigned to various manifestations of sequence variation, such as, insertions, deletions, substitutions, etc.

The invention will now be illustrated with reference to certain examples which are included herein for the purposes of illustration only, and which are not intended to be limiting of the invention.

### EXAMPLE 1

### Materials and Methods—Study 1

Plasmid Construction and Mutagenesis.

Plasmid pMono expresses wild-type or mutated monomeric protease from the tac promoter (Amann et al. (1983) Gene 25:167-178) in Escherichia coli and was derived from plasmid P1+IQ (Baum et al. (1990) Proc. Natl. Acad. Sci. USA 87:10023-10027). Protease sequences in P1+1Q were replaced by a linker of 5′ XhoI-XbaI-SalI-PstI 3′ to produce p1CVx. Wild-type or mutated protease sequences were obtained from pET-PR by PCR with primers 5′Xho (AATATA-CTCGAG-GAAGGAGATATACAT, SEQ ID NO: 1) and 3′Xba (ATAAAT-TCTAGA-CTTGGGCTGCAGGG, SEQ ID NO: 2) and were inserted into p1CVx to produce pMono. The phagemid pET-PR contains the 99-residue coding domain of the monomeric protease (HXB2 isolate [Ratner et al. (1987) AIDS Res. Hum. Retrovir. 3:57-69]) inserted into the NdeI-BamHI sites of pET24a (Novagen). The Kunkel method using single-stranded templates of pET-PR substituted with uracil was employed for site-directed mutagenesis (Bebenek and Kunkel (1989) Nucleic Acids Res. 17:5408, Kunkel et al. (1991) Methods Enzymol204:125-139). Mutations were confirmed by DNA sequencing prior to transfer of the protease sequences into p1CVx.

The phagemid pGag1 was the parent construct of both pGPfs and pGPfs-PR. pGag1 contains the upstream leader, Gag, and Pol sequences from HIV-1 isolate HXB (GenBank accession no. NC 001802; Ratner et al. (1987) AIDS Res. Hum. Retrovir. 3:57-69) from the NarI site (base 182) to the SalI site (base 5331) inserted into the XbaI and SalI sites of pIBI20 (International Biotechnologies) downstream of the T7 promoter of pIBI20. The frameshift mutation in pGagFS was constructed by site-directed mutagenesis of uracil-substituted single-stranded pGag1 as described above with the following oligonucleotide: GAG AGA CAG GCT MC TTC CTC CGC GAA GAC TTG GCC TTC CTA CM GGG (SEQ ID NO: 3). The frameshift mutation, when translated, reproduces precisely the amino acid sequence of the major GagPol Pr160 product (Gorlick and Henderson (1994) Part III: analyses, p. 2-5. In G. Myers, B. Korber, S. Wain-Hobson, K. T. Jeang, L. Henderson, and G. Pavlakis (ed.) Human retroviruses and AIDS Los Alamos National Laboratory, Los Alamos, N. Mex., Jacks et al. (1988) Nature 331:280-283). pGPfs-PR was constructed from pGPfs by a D25A substitution of the catalytic aspartate of the protease. Further mutations within pGPfs and pGPfs-protease were introduced in the respective plasmids by the same method.

Western Blot Determination of the Percentage of β-Galactosidase Cleavage in E. coli.

Uninduced mid-log-phase (optical density at 600 nm=0.5) cultures of lac-E. coli strain MC1061 carrying pMono were grown in yeast-tryptone medium to produce samples for Western blot analysis. Cells were pelleted, resuspended in 400 μl of sodium dodecyl sulfate (SDS)-polyacrylamide gel electrophoresis (PAGE) sample buffer (Pettit et al. (1991) J. Biol. Chem. 266:14539-14547) (optical density at 600 nm=˜0.5), and heated to 95° C. for 4 min. SDS-PAGE was performed on Tris-glycine gels (7.5% polyacrylamide) (Laemmli (1970) Nature 277:680-685). Separated proteins were bound to nitrocellulose by electrotransfer, blocked for 1 h with 3% bovine serum albumin in Tris-buffered saline-0.1% Tween 20, and probed with anti-β-galactosidase monoclonal antibody (Boehringer) at a 1:5,000 dilution. Following multiple washes in Tris-buffered saline-0.1% Tween 20, β-galactosidase was detected with the ECL Plus system followed by autoradiography according to the instructions of the manufacturer (Amersham Pharmacia). The extent of β-galactosidase cleavage was determined by densitometric measurement on a Molecular Dynamics Storm model 800 Phosphorlmager. For scanning, the Phosphorlmager was set in the blue fluorescence and chemifluorescence mode with a photomultiplier tube voltage of 700 V. The final value for percent cleavage of the β-galactosidase substrate was determined by taking the average of two or more independent inductions.

In vitro Assays of the Proteolytic Processing of Gag.

Transcription and translation of pGPfs or pGPfs-PR was performed in 50-μl reaction volumes with rabbit reticulocyte lysate (RRL) and 20 μCi of [³⁵S]cysteine (>1,000 Ci/mmol; Amersham Pharmacia Biotech), using the TNT system (Promega). For cis protease processing reactions, 5-μl aliquots from the pGPfs translation reaction mixtures were taken at indicated times and the reaction was stopped by the addition of 10 μl of lithium dodecyl sulfate (LDS)-PAGE loading buffer (Invitrogen).

For trans protease processing reactions, transcription and translation of pGPfs-PR-based constructs proceeded for 2 h at 30° C. trans processing of the Gag precursor derived from pGPfs-PR was performed in 50-μl reaction volumes containing 5 μl of RRL and 0.25 to 0.5 μg of purified recombinant protease in phosphate buffer (25 mM Na₂HPO₄, 25 mM NaCl, and 1 mM dithiothreitol, pH 7.0). Reactions were performed for 4 h at 30° C. Five-microliter aliquots were removed at various times, and the reaction was stopped by the addition of an equal volume of 2× LDS-PAGE loading buffer (Invitrogen). Products of the processing reaction were heated to 70° C. prior to separation on NuPage Bis-Tris gradient gels (4 to 12% polyacrylamide) as recommended by the manufacturer (Invitrogen). Gels were fixed in 10% acetic acid and dried prior to performance of autoradiography.

Expression and Purification of HIV Protease (PR).

Recombinant wild-type HIV protease (PR) was expressed in E. coli and purified and refolded as described previously (Gulnik et al. (1995) Biochemistry 34:9282-9287). Briefly, the cells were resuspended in buffer A (50 mM Tris-HCl buffer [pH 8.0], 25 mM NaCl, 0.2% β-mercaptoethanol), sonicated, and centrifuged. Inclusion bodies were washed first with buffer A, then with buffer A containing (consecutively) 0.1 % Triton X-100, 1 M NaCl, and 1 M urea, and finally with buffer A alone. Purified inclusion bodies were solubilized by addition of buffer A containing 8 M urea at room temperature. The solution was clarified by centrifugation and loaded onto a 2.6- by 9.5-cm Q-Sepharose column equilibrated with 8 M urea in buffer A. Flowthrough fractions were collected and dialyzed against three changes of refolding, buffer, which consisted of 25 mM sodium phosphate (pH 7.0), 25 mM NaCl, 0.2% β-mercaptoethanol, and 10% glycerol. The total yield of the purified protease was 5 to 10 mg/liter of E. coli culture. The percentage of active sites in the prepared protease was determined by active-site inhibition and titration (Tomasselli et al. (1990) Biochemistry 29:264-269) with the tight-binding inhibitor ABT-538 (Gulnik et al. (1995) Biochemistry 34:9282-9287). ABT-538 inhibited cleavage of GagPol (PR negative) by 400 nM trans-protease (by monomer weight) at a 50% inhibitory concentration of approximately 100 nM, indicating that >50% of the protease was present in the active, dimeric form (data not shown).

### EXAMPLE 2

### Results—Study 1

Expression of the GagPol Precursor in vitro Results in Protease Activation and Precursor Cleavage.

The HIV-1 Gag and Pol coding domains are overlapping; gag encodes the structural genes of the viral core (from the 5′ end: matrix [MA], capsid [CA], p2, nucleocapsid [NC], and p6), and pol encodes viral enzymes, including protease (PR), reverse transcriptase (RT), and integrase (IN) (Orozan and Luftig (1990) Curr. Top. Microbiol. Immunol. 157:153-185). The GagPol precursor is a fusion protein that is translated as the result of a -1 frameshift near the end of the gag gene (Jacks et al. (1988) Nature 331:280-283, Reil et al. (1993) J. Virol. 67:5579-5584).

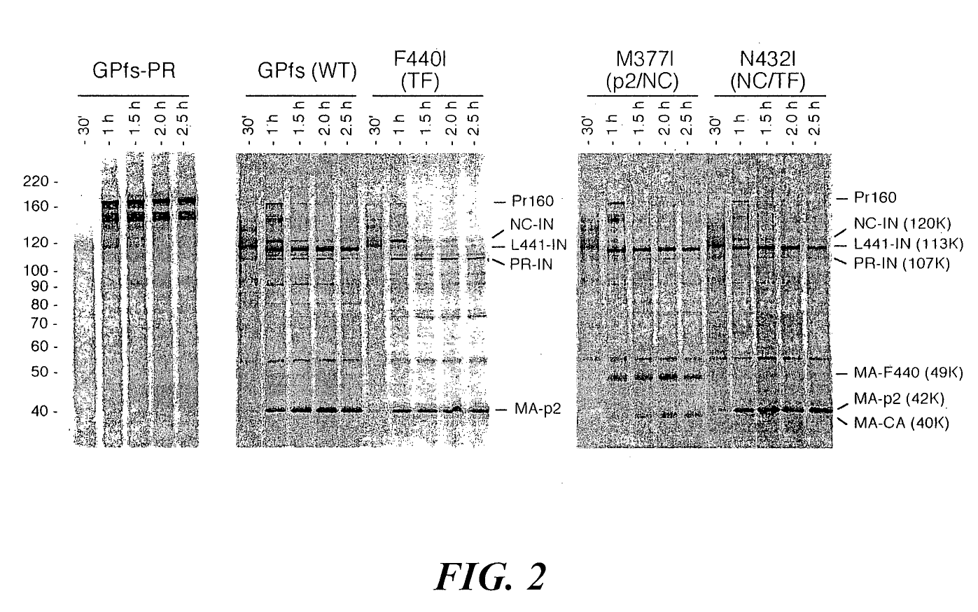

To assess the activation of the immature protease in the context of the precursor and to characterize the determinants controlling precursor processing, an HIV-1 GagPol rabbit reticulocyte lysate (RRL) expression system was developed in which the precursor is cleaved by endogenous protease. We introduced a forced frameshift mutation in the gag and pol open reading frames (FIG. 1, pGPfs) to reproduce, exactly in sequence, the major GagPol frameshift product seen in cells infected by HIV-1 (Gorelick and Henderson (1994) Part III: analyses, p. 2-5 In G. Myers, B. Korber, S. Wain-Hobson, K. T. Jeang, L. Henderson, and G. Pavlakis (ed.) Human retroviruses and AIDS Los Alamos National Laboratory Los Alamos, N. Mex.). This construct expresses full-length GagPol Pr160 as evidenced by SDS-PAGE (FIG. 1). Translation of the pGPfs construct containing active protease in RRL resulted in the transient appearance of the full-length Pr160 GagPol precursor at approximately 1 h into translation (FIG. 2). This was followed by the transient appearance of a 120-kDa primary intermediate before the generation of 113- and 41-kDa products (FIG. 2, 3 and 4; see also FIG. 6). Beyond 2 h of translation, there was no further accumulation of precursors or final products. A construct expressing full-length GagPol with an alanine substitution for the catalytic aspartic acid (pGPfs-PR) produced only the expected Pr160 product. This was stable over 2.5 h of translation (FIG. 2). A number of minor bands that likely represent internal initiations or premature terminations during translation were observed.

The precise location of the primary and secondary processing sites was determined by placing blocking mutations at individual cleavage sites; it has been demonstrated that substitution of an lie for the P1 residue of a substrate severely inhibits cleavage (Billich et al. (1988) J. Biol. Chem. 263:17905-17908, Pettit et al. (1994) J. Virol. 68:8017-8027, Tozser et al. (1992) Biochemistry 31:4793-4800). We inserted blocking mutations into two known protease-processing sites, p2/NC (M377I) and NC/p1 (N432I) (Henderson et al. (1990) J. Med. Primatol. 19:41 1-419, Wondrak et al. (1993) FEBS Lett. 333:21-24). In addition, we substituted an lie at a novel Phe/Leu site previously reported in Almog et al. (1996) J. Virol. 70:7228-7232, located 8 residues downstream from NC in the transframe (TF) domain of GagPol (F4401) (FIG. 1).

We found that initial cleavage of wild-type GagPol occurred at the p2/NC site, generating the 42-kDa MA-CA-p2 intermediate and the 120-kDa NC-TF-PR-RT-IN product. The 120-kDa product is further cleaved at TF F440/L441 to generate the 113-kDa TF L441-PR-RT-IN product. Blocking the initial p2/NC site (M377I) resulted in the appearance of alternative products composed of MA-CA (40-kDa) and MA-CA-p2-NC-TF F440 (49-kDa) in addition to the TF L441-PR-RT-IN (113-kDa) product (FIG. 2). Likewise, an F440I substitution prevented accumulation of the secondary 113-kDa product (TF-PR-RT-IN) and extended the presence of the initial 120-kDa product (NC-TF-PR-RT-IN). The absence of the 49-kDa product (indicative of cleavage at the TF F440/L441 site) and the predominance of the 42-kDa product (indicative of cleavage at p2/NC) during early cleavage of wild-type GagPol are consistent with primary cleavage of the p2/NC site (FIG. 2, GPfs, 1-h time point).

Blocking the NC/TF site had no noticeable effect on the generation of products, suggesting that this site is not among those cleaved. The identity of the cleaved sites was confirmed by excising two residues flanking the scissile bond (P1-P1′) of either site, making the site unrecognizable by the protease. The cleavage pattern obtained with these constructs was the same as that which we observed when we were using the lie cleavage site-blocking mutations (data not shown). Of note, initial cleavage at the p2/NC site has been observed in Gag processing in HIV-infected cells (Pettit et al (1994) J. Virol. 68:8017-8027, Shehu-Xhilaga et al. (2001) J. Virol. 75:9156-9164, Wiegers et al. (1998) J. Virol. 72:2846-2854). This result is also consistent with studies of Gag trans processing in which the p2/NC site was cleaved most rapidly (Carter and Zybarth (1994) Methods Enzymol. 241:227-253, Erickson-Vitanen et al. (1989) AIDS Res. Hum. Retrovir. 5:577-591, Kräusslich et al. (1988) J. Virol. 62:4393-4397, Pettit et al. (1994) J. Virol. 68:8017-8027).

When either the p2/NC or the TF F440/L441 site was blocked, increased cleavage of alternative neighboring sites was observed. For example, blocking the p2/NC site produced a novel 40-kDa product (MA-CA), indicating alternate site selection of the CA/p2 site, which is not normally cleaved in this assay. Similarly, blocking cleavage of the TF F440/L441 site resulted in increased amounts of the 107-kDa PR-RT-IN intermediate compared to those of wild-type GagPol, indicating enhanced cleavage at the N terminus of the protease.

Individual Residues of the Dimer Interface Play Differential Roles in Protease Activation.

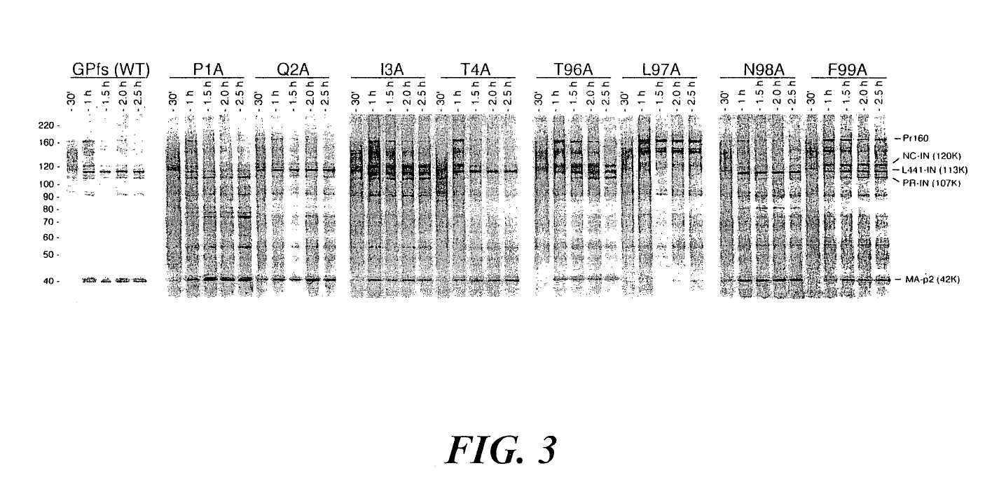

Given the relationship between protease dimerization within GagPol and enzyme activation, we assessed the role of individual dimer interface residues in protease activation. We performed alanine-scanning mutagenesis on residues 1 to 4 and 96 to 99-of the protease dimer interface in the GagPol expression system and examined the impact of these substitutions on enzyme activation and specificity. These results were later compared with data obtained by substitution of alanines for dimer interface residues in the mature 99-amino-acid protease. As shown in FIG. 3, individual alanine substitutions had differential effects on activation. Substituting alanine for residue 2, 4, or 98 (Gln, Thr, or Asn, respectively) had little effect on protease activation compared to wild-type GagPol (GPfs). Complete cleavage of the Pr160 precursor occurred roughly 1.5 h into the translation reaction. The L97A substitution resulted in the most severe defect in activation, as there was no observed cleavage of the Gag precursor during the 2.5-h assay. The I3A, T96A, and F99A substitutions resulted in reduced activity and incomplete cleavage of the precursor. Decreased protease activation in these three mutants resulted in increased amounts of the initial 120-kDa NC-TF-PR-RT-IN product, which is typically cleaved further to the 113-kDa TF F441-PR-RT-IN product in wild-type GPfs. In addition, increased amounts of the 107-kDa PR-RT-IN intermediate were seen, indicative of increased cleavage of the TF/PR site. However, none of the mutations tested increased the rate of cleavage of the PR/RT site, as the 97-kDa RT-IN product remained minor.

The P1A substitution resulted in a protease that appeared to be fully active,_as judged by the disappearance of the Pr160 precursor, but with altered specificity toward distal sites. Products in the range of 75 to 95 kDa were generated in addition to the typical 120-kDa and 113-kDa intermediates formed from initial and secondary cleavage of the p2/NC and TF F440/L441 sites, respectively. There was also enhanced cleavage at the TF/PR site at the amino terminus of the protease.

We compared these results with the effect of the same alanine substitutions in the context of the mature 99-amino-acid protease molecule. For these studies, we modified an E. coli expression system developed as described in Baum et al. (1990) Proc. Natl. Acad. Sci. USA 87:10023-10027. The vector (pMono) expresses HIV protease and the β-galactosidase gene. A cleavage cassette consisting of a decapeptide corresponding to an HIV protease cleavage site was inserted into the β-galactosidase open reading frame. In this system, enzyme activity may be assessed via Western blotting as the percent cleavage of full-length β-galactosidase.

Again, single alanine substitutions in the monomeric protease produced differential effects on activity (Table 1). As in the full-length-GagPol activation assay, we found that P1A, Q2A, and T4A substitutions had no effect on protease activity. Introducing an alanine at position 3, 97, or 99 completely inhibited protease activity. The N98A mutation showed an intermediate phenotype with 68% cleavage of the substrate. Our results suggest that there are significant differences between the effects of identical mutations on the activity of the GagPol protease and the results obtained with the mature protease. Overall, mutations had less of an effect on enzyme activity when present in the precursor form. I3A and F99A were inactive in the processed protease and intermediate in the GagPol-processing system. Similarly, increases in enzyme activity were seen when the T96A and N98A substitutions were expressed in the full-length construct. This comparison suggests that sequences within GagPol can compensate, to some degree, for detrimental mutations in the dimer interface.

TABLE 1
Effect of single alanine substitutions of the interface residues
on the activity of processed PR on cleavage of a β-galactosidase
substrate in E. coli

| Mutation | % Cleavage |
|---|---|
| Wild type | 96 |
| D25A* | 0 |
| P1A | 92 |
| Q2A | 96 |
| I3A | 0 |
| T4A | 96 |
| T96A | 14 |
| L97A | 0 |
| N98A | 68 |
| F99A | 0 |

^{a}D25A is a replacement of the catalytic aspartate and serves as a negative control.

The Proline at Position 1 of the Protease Serves as a Determinant of Specificity for the Activated Protease.

The substitution of alanine for proline at position 1 of the protease produced enhanced cleavage of the amino terminus of the enzyme. In addition, this substitution uniquely altered the specificity of the order of GagPol cleavage without greatly affecting overall enzyme activity. We introduced other substitutions at position 1 (Gly, Phe, and Leu) to examine the influence of this residue on enzyme activation and specificity. All substitutions tested resulted in similarly altered specificity without greatly affecting the overall activity of the protease. Altered specificity resulted in decreased amounts of the 113-kDa TF L441-PR-RT-IN product and increased amounts of other products in the range of 60 to 95 kDa. Although the degree of altered specificity varied with each substitution, the generation of products is similar for the different substitution mutants (FIG. 4, right-facing arrows). P1A, P1F, and P1L also increased relative amounts of the 107-kDa PR-IN product, indicating enhanced cleavage at the amino terminus of the protease (TF/PR). The P1G substitution differed only in that it inhibited cleavage at the TF/PR (FIG. 4, left-facing arrow). These results suggest that Pro 1, as a component of activated GagPol protease, functions as a specificity determinant for the cleavage of distal sites in addition to influencing the rate of cleavage at the TF/PR site.

Mutations in the Protease Dimer Interface Alter the Pattern of Cleavage of the GagPol Precursor when Purified Protease is Added in Trans.

The processing of the GagPol precursor was also evaluated with purified protease provided in trans. Full-length GagPol (pGPfs) was modified by a D25A substitution at the protease active site (pGPfs-PR), and the kinetics of trans processing was monitored over time by the addition of purified recombinant wild-type protease.

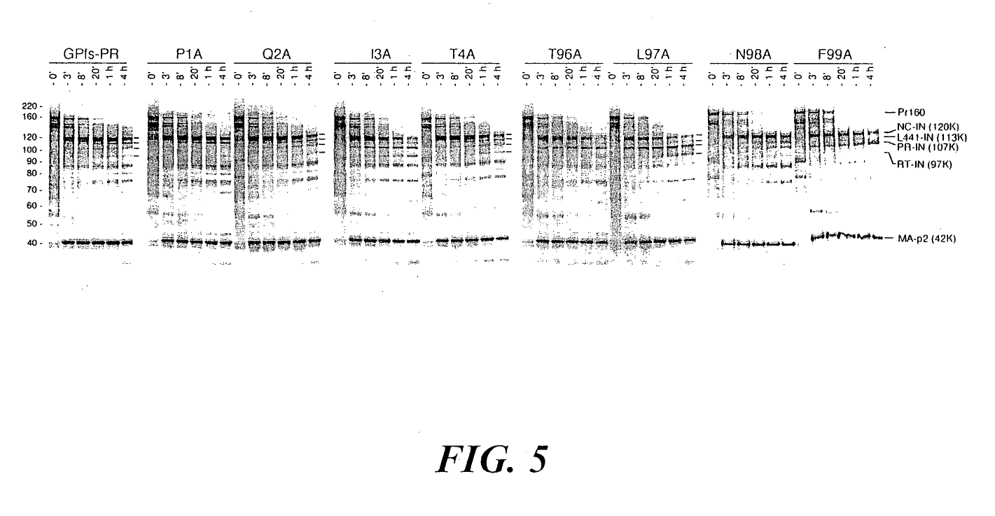

Precursor processing of wild-type Gag Pol by trans-protease occurred in an order similar to that observed in cis processing. Initial cleavage occurred rapidly at the p2/NC site (FIG. 5, 120-kDa product) followed sequentially by cleavage at the TF F440/L441 site (113-kDa product). Cleavage at these sites was confirmed by the disappearance of the appropriate intermediates upon introduction of the M377I and F440I blocking mutations (data not shown). As reported above with the expression of the pGPfs construct, little processing was observed at the sites at the N and C termini of the protease itself when purified enzyme was added in trans (FIG. 5, 107- and 97-kDa products).

Residues 1 to 4 and 96 to 99 were individually replaced with alanines in the pGPfs-PR construct, and the effect on processing was assessed. The majority of the mutations increased cleavage of the sites flanking the protease and decreased cleavage of the TF F440/L441 site. Increased cleavage at the amino terminus of the protease was most pronounced with replacement by alanine of the isoleucine normally found at position 3 of the protease and with the carboxy-terminal T96A, L97A, and F99A substitutions. Enhanced cleavage of the protease carboxy terminus was most pronounced with the I3A and L97A substitutions. The Q2A and T4A substitutions showed little effect on trans-processing kinetics. As expected, the Phe-to-Ala substitution at position 99 prevented processing at the carboxy terminus of the protease; this result is likely due to the unfavorable effect of an alanine at the P1 position of the substrate.

trans processing of the constructs containing substitutions at position 1 of the protease differed dramatically from the results obtained when the precursor was processed by the endogenous GagPol protease. For the P1 A mutant, we observed an altered pattern of processing when the precursor was cleaved by the endogenous protease within GagPol (FIG. 3 and 4). In contrast, adding purified protease in trans to the pGPfs-PR construct containing the P1A substitution produced a cleavage pattern largely indistinguishable from that of the wild-type GagPol (FIG. 4). Further, cleavage patterns similar to the parental pGPfs-PR construct were seen when the protease was added in trans to the P1G, P1F, or P1L substitution (data not shown). It is important that, because a wild-type cleavage pattern was observed when the purified protease was delivered in trans, it is unlikely that the altered pattern of pGPfs cleavage obtained with the mutant P1A (FIG. 3) is due to a global disordering of the precursor structure.

Mutation of the Amino Acids Immediately N and C to the Protease Suggests that these Flanking Residues do not Play a Role in Enzyme Activation.

Our alanine-scanning comparisons between the mature protease and the GagPol-associated protease suggest that, when the protease is embedded within GagPol, there are alternate domains outside the protease that contribute to enzyme dimerization and activation. It is possible that the protease dimer interface itself (i.e., residues 1 to 4 and 96 to 99) extends beyond the amino and carboxy termini of the protease into the adjoining GagPol coding domains. To define the borders of the protease dimer interface, we produced GagPol constructs in which the five amino acids N terminal or the five amino acids C terminal to the protease domain were replaced with alanine.

These substitutions had little effect on the kinetics or specificity of activation compared to wild-type GPfs (FIG. 6). This results suggests that the side chains of the five residues immediately flanking the protease in GagPol have little influence on activation of the protease and initial cleavage of the precursor. As expected, the N-terminal substitutions prevented cleavage at the TF/PR site, as the 107-kDa PR-RT-IN minor product was absent in the TF 5A/PR mutant (FIG. 6, center, double arrow). Similarly, replacing the five residues immediately downstream of the protease with alanine (PR/5A RT) showed little effect on protease activation. The 97-kDa RT/IN product was not observed in either the wild-type GPfs or the PR/5A RT constructs, indicating that the PR/RT site is not cleaved in either context.

### EXAMPLE 3

### Material and Methods—Study 2

Plasmid Construction and Mutagenesis.

The construction of pGPfs and pGPfs-PR was as described above in Example 1. HIV-1 sequences were derived from an HXB isolate of HIV-1 (accession NC 001802; Ratner et al. (1987) AIDS Res. Hum. Retrovir. 3:57-69). Briefly, pGPfs contains a single GagPol open reading frame downstream of the bacteriophage T7 promoter in vector pIBI20 (International Biotechnologies). PGPfs-PR contains an additional catalytic mutation (D25A) of the protease domain that renders PR inactive. In both plasmids, a continuous GagPol open reading frame was created by site-directed mutagenesis-to reproduce exactly in amino acid sequence the major GagPol product found in virions (Gorelick and Henderson (1994) Part III: Analyses, p. 2-5. In G. Myers and B. Korber and S. Wain-Hobson and K. T. Jeang and L. Henderson and G. Pavlakis (ed.), Human Retroviruses and AIDS. The Los Alamos National Laboratory, Los Alamos, N. Mex., Jacks et al. (1988) Nature 331:280-283). pGag1 contains the Gag and Pol open reading frames and produces full length pr55 Gag and pr160 GagPol in an approximate 20:1 ratio by translational frameshift during translation in vitro (see Examples 1 and 2 above). pGagS was previously described and produces only the pr55 Gag product upon translation in vitro (Pettit et al. (2002) J. Virol. 76:10226-10233, Pettit et al. (1994) J. Virol. 68:8017-8027). Site directed mutagenesis was performed as described (Bebenek and Kunkel (1989) Nucleic Acids Res. 17:5408, Kunkel et al. (1991) Methods Enzymol. 204:125-139). All mutations were confirmed by direct sequencing by the dideoxy-termination method prior to use.

In vitro Assays for the Proteolytic Processing of Gag.

Transcription and translation of pGPfs, pGPfs-PR, pGag1, or pGag was performed in rabbit reticulocyte lysate (RRL) using the TNT system (Promega) in 50 μl reactions with 20 μCi of [³⁵S] cysteine (>1000 ci/mM Amersham Pharmacia Biotech). For co-expression of Gag/GagPol or GagPol/GagPol containing various mutations, plasmids were premixed prior to transcription/translation to express the respective protein products in ratios according to the molecular mass of the products. The concentration of all products expressed in RRL is estimated at approximately 1 nM (data not shown).

For cis-protease processing reactions, 5 μl aliquots from the pGPfs translation reactions are taken at indicated times and stopped by the addition of 10 μI LDS-polyacrylamide gel electrophoresis (LDS-PAGE) loading buffer (Invitrogen). For trans-protease processing reactions, transcription and translation of pGPfs-PR based constructs proceeded for 2 h at 30° C. Trans-processing reactions of the GagPol precursor derived from pGPfs-PR were performed in 50 μl reactions containing 3 μl of RRL and 400 nM purified recombinant protease in phosphate buffer (25 mM Na₂HPO₄, 25 mM NaCl, 1 mM DTT, pH 7.0). Reactions were performed for 4 h at 30° C. 5 μl aliquots were removed at various times and stopped by the addition of an equal volume of 2× LDS-PAGE loading buffer (Invitrogen). Products of the processing reaction were heated to 70° C. prior to separation on NuPage 4-12% Bis-Tris gradient gels as recommended by the manufacturer (Invitrogen). Gels were fixed in 10% acetic acid and dried prior autoradiography and captured for densitometric analysis on a Molecular Dynamics Storm model 800 phosphoimager.

Competitive Inhibition of cis- and trans-Protease Processing Reactions by Ritonavir.

Ritonovir (ABT538-Abbot) was serially diluted in 20% w/w dimethyl sulfoxide (DMSO)/water to provide a 20× stock of inhibitor (1% DMSO final concentration). For cis-protease processing reactions, transcription and translation was performed as described above in 20 μl total volume with 1 μl of 20× ritonavir stock added prior to transcription and translation. The trans-protease processing reactions were carried out as above with 1 μl volume 20× ritonavir stock added prior to the addition of purified protease (PR). Reactions were preincubated for 10′ on ice prior to the addition of purified PR to start the reaction. Reactions were incubated for 20′ to 1 hr at 30° C. IC₅₀ values for the inhibition of the p2/NC (M377/M378) and TF F440/L441 sites were calculated as follows: first, densitometic analysis was performed on respective gels and relative protein ratios for precursors and products were calculated based on their known composition and the number of labeled cysteines residues of each. IC₅₀ values were estimated from the determination of percent uncleaved substrate as calculated from the ratio of product vs. precursor at each concentration. An additional normalization was used to set the percentage of uncleaved substrate to 0% in the control reaction with no added inhibitor. The concentration of inhibitor necessary for 50% inhibition of each site was extrapolated from plots of the percent uncleaved substrate vs inhibitor concentration. This procedure for the estimation of IC₅₀ values is a modification of an earlier procedure previously used to estimated the differences in the rate of cleavages of HIV-1 Gag processing sites (Pettit et al. (2002) J. Virol. 76:10226-10233, Pettit et al. (1994) J. Virol. 68:8017-8027).

Expression and Purification of HIV PR.

Recombinant wild type HIV PR was expressed in E. coli purified from inclusion bodies, and refolded as described previously (Gulnik et al. (1995) Biochemistry 34:9282-9287; Example 1 above). Total yield of purified protease was 5-10 mg/liter of E. coli culture in a buffer consisting of 25 mM Na-phosphate, pH 7.0, 25 mM NaCl, 0.2% β-mercaptoethanol and 10% glycerol. Final concentration of PR was 0.1 mg/ml before storage at −70° C.

### EXAMPLE 4

### Results—Study 2

Expression of the Full Length GagPol Precursor Results in Processing by the Endogenous PR.

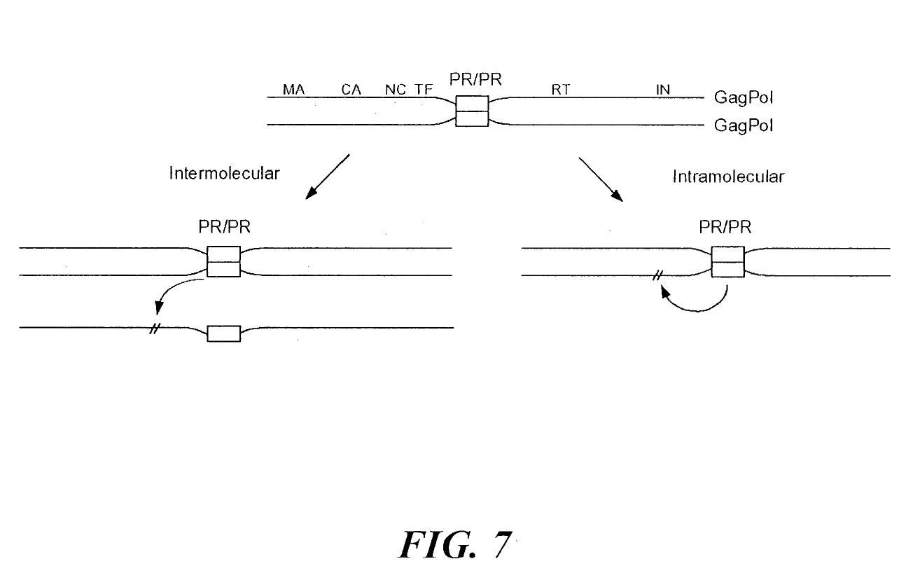

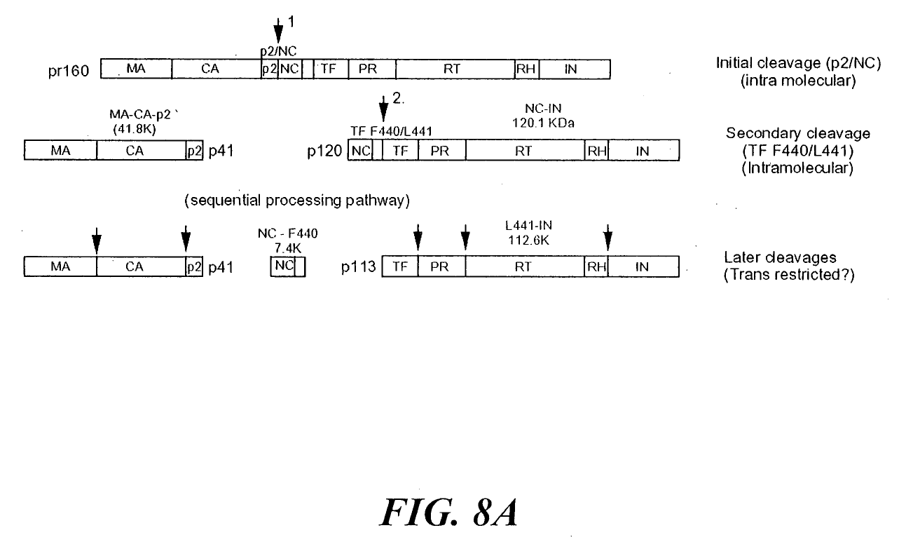

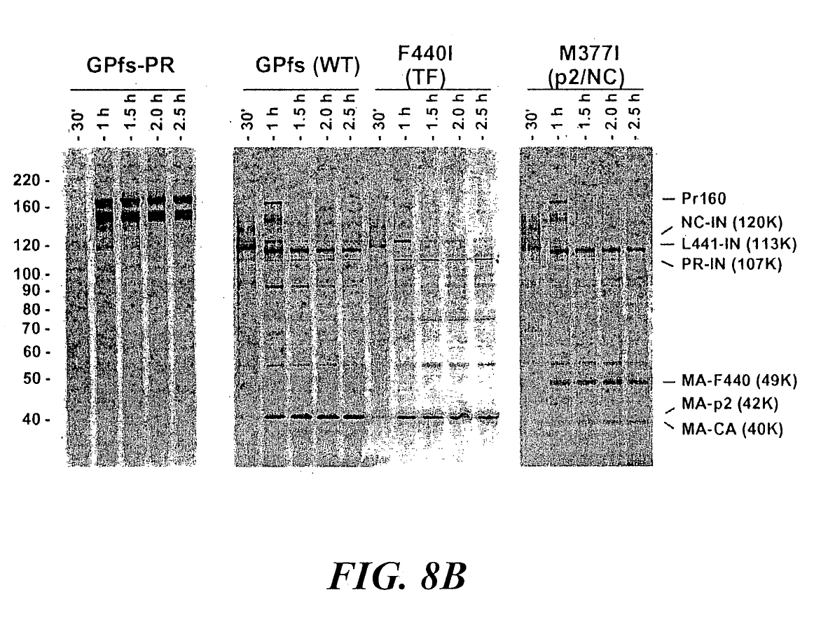

The processing of a full-length GagPol precursor by its endogenous PR was examined in a rabbit reticulocyte lysate (RRL) system in order to evaluate the earliest steps in PR activation and GagPol cleavage. For example, it is uncertain whether the initial cleavages mediated by the protease are inter- or intra-molecular (FIG. 7). Expression of the full length GagPol precursor in a rabbit reticulocyte lysate system resulted in ordered processing of the first two processing sites (p2/NC M377/378 and transframe F440/L441) by the embedded viral PR (Example 2 above). Three fragments are produced: a 42 kDa fragment containing the MA, CA and p2 proteins, a small (7.4 kDa) protein consisting of the viral NC with an eight amino acid C-terminal extension, and a 113 kDa protein containing the transframe region, the PR itself, the RT and IN (see schematic in FIG. 8A). To identify the cleavage sites, the observation that the substitution of a β-branched amino acid at the P1 position of a PR cleavage site blocks cleavage at that site was considered (Pettit et al. (2002) J. Virol. 76:10226-10233, Pettit et al. (1994) J. Virol. 68:8017-8027, Pettit et al. (1991) J. Biol. Chem. 266:14539-14547, Tozser et al. (1992) Biochemistry 31 :4793-4800) by HIV-1 protease (the P1 position is the first residue upstream of the scissile bond; Schechter and Berger (1967) Biochem. Biophys. Res. Commun. 27:157-162). GagPol containing these substitutions produce a readily distinguishable cleavage pattern (Example 2 above) that indicted that the substituted site was blocked for cleavage. Alternative site selection was also noted when the preferred site is blocked. The alternative site chosen was close in proximity to the preferred site, and was a site not typically cleaved by activated GagPol protease in vitro. For example, the Phe to lie substitution at position 440 blocked cleavage within the transframe region and enhanced cleavage at the amino-terminus of the PR (FIG. 8B). In this case the 113 kDa intermediate seen with expression of the wild type GagPol precursor was replaced by a smaller 107 kDa species that contains PR, RT and IN (FIG. 8B, F440I). Similarly, the introduction of a Met to lie substitution at amino acid 377 of the p2/NC M377/M388 cleavage site resulted in the disappearance of the expected band at 42 kDa (MA-CA-p2) (FIG. 8B, M377I). In its place, a smaller, 40 kDa band representing enhanced cleavage at an alternate site upstream (CA/p2 L363/A364) was seen (FIG. 8B). These results indicate that if the preferred PR processing site is blocked, cleavage at a neighboring site, typically not cleaved this early, may occur.

The PR embedded within GagPol is Relatively Insensitive to Inhibition by an Active Site Inhibitor.

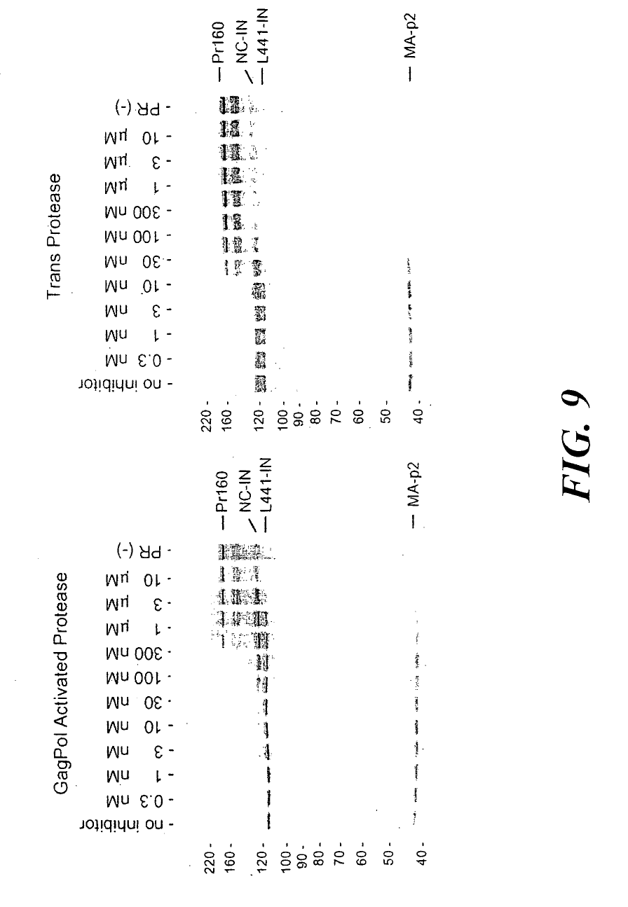

This full-length GagPol processing system was used to characterize the sensitivity of the GagPol PR to an active site inhibitor (ritonavir). Specifically, processing of GagPol by the embedded PR in the presence of increasing concentrations of ritonavir was examined. Each site was inhibited to different degrees in response to increasing ritonavir concentration, with the slower cleaved site being inhibited first (FIG. 9, Left Panel). 50% inhibition of processing occurs at approximately 644 nM ritonavir for the slower TF F440/L441 site and 8.25 μM of ritonavir for the faster p2/NC site (FIG. 9, Left Panel). These values are significantly higher than the sub-picomolar inhibitor constant (Ki) values for ritonavir derived from purified, processed protease (Klabe et al. (1998) Biochemistry 37:8735-8742, Molla et al. (1996) Nature Med. 2:760-766).

A direct comparison was sought between the sensitivity of the mature PR to inhibition by an active site inhibitor with the results obtained with the embedded PR utilizing GagPol as a substrate. This trans-processing reaction was accomplished by adding purified PR in trans to a GagPol construct encoding a PR inactivated by an Asp to Ala substitution at the enzyme active site (GagPol PR−). In contrast to the results obtained above with an active embedded PR (FIG. 9, Left Panel), exogenously added PR was much more sensitive to inhibition by ritonavir (FIG. 9, Right Panel). For the purified PR processing the full-length GagPol PR− in trans, cleavage at p2/NC is 50% inhibited at a ritonavir concentration of 107nM and the TF F440/L441 site at 18 nM (FIG. 9, Right Panel). Of note, for these studies, although the concentration of wild type GagPol (and, therefore, the endogenous PR) is approximately 1 nM, 400 nM of PR is added to the GagPol PR− construct in order to see processing. Therefore, processing of the precursor by the embedded protease is approximately 10,000-fold less sensitive to inhibition by this active site inhibitor than is processing of the precursor in trans by purified, mature protease.

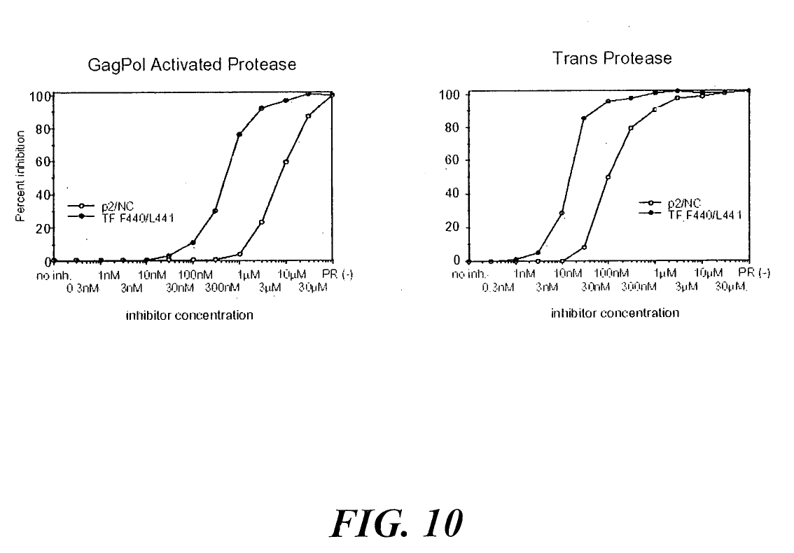

The concentration of ritonavir required to inhibit cleavage at two different sites in GagPol (M377/M378 and F440/L441) was also compared. The concentration of ritonavir required to inhibit cleavage by 50% was found to differ by more than 20-fold for the two sites (8.25 μM vs. 400 nM) (FIG. 10, left panel). Once again, in contrast to the results obtained with the endogenous PR, ritonavir inhibition of purified PR added in trans differed by only 5.9-fold across the two cleavage sites (18 nM vs. 107 nM; FIG. 10, right panel).

The Initial Cleavages within the GagPol Precursor are Intramolecular.

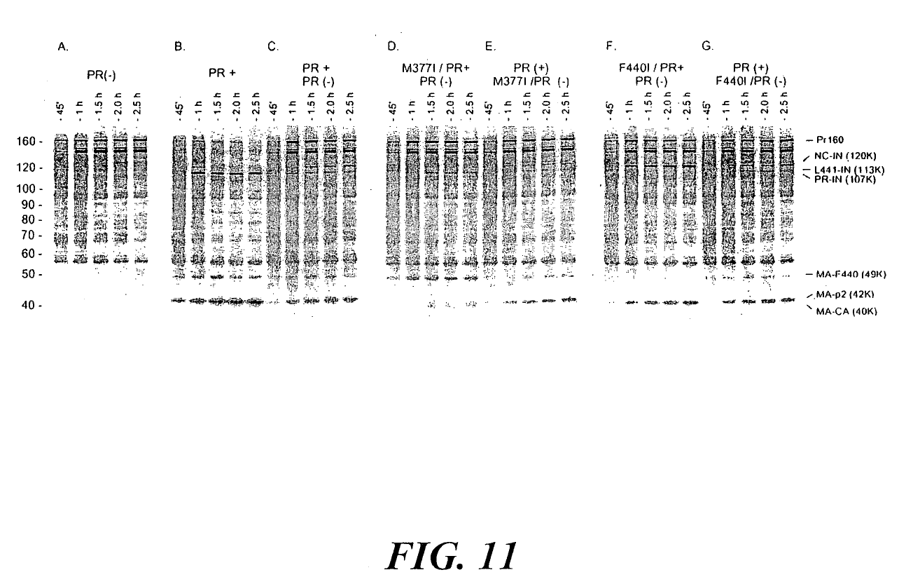

The extent of intra- vs. intermolecular processing of the full-length GagPol further was characterized through mixing experiments in which equivalent amounts of substituted GagPol constructs were co-expressed. In these experiments, GagPol constructs encoding various combinations of PR active site substitutions were cotranslated (GagPol PR−) and/or processing site mutations (M377I and F440I) (FIG. 11). These experiments took advantage of the observation that the introduction of these processing site substitutions produces distinct cleavage patterns.

In summary, it was observed that the pattern of cleavage was dictated by the construct containing the active PR domain: (FIG. 5). Overall, it was found that co-expression of GagPol constructs that contained an active PR and wild type processing sites with a construct that contained an inactivated PR and a blocked processing site showed a wild type pattern of cleavage. Alternatively, if the active PR domain was present on the construct with a mutated cleavage site, the altered cleavage pattern was observed (FIG. 11). For example, expression of a GagPol construct containing an active PR and a Met to lie blocking substitution at position 377 (M377I/PR+) together with a GagPol construct containing an inactive PR and a Met at position 377 (PR−) (FIG. 11, Panel D). The observed pattern was similar to the pattern seen when the GagPol with M337I substitution was expressed alone (FIG. 8B). In contrast, co-expression of the wild type GagPol construct (PR+) with a construct containing the M377I substitution and the D25A PR substitution (M377I/PR−) resulted in a wild type pattern of cleavage (FIG. 11, Panel E). Similar results were obtained when constructs containing combinations of the Phe to IIe substitution at position 440 with either active or inactive PR were co-expressed (FIG. 11, Panels F and G). In all cases, a wild type cleavage pattern was observed for the constructs with wild type cleavage sites in which the PR was intact and an altered pattern of processing was observed when the intact PR active site was paired with a mutated cleavage site on the same construct (FIG. 11).

Of note, overall processing was significantly diminished by the co-expression of a construct which contained a wild type PR with a construct containing an active-site substituted PR (FIG. 11, Panel C, PR+/PR−). If the mutated protease was exerting a true trans-dominant inhibitory effect, absence of protease activity would be expected in 75% of the GagPol expressed due the formation of PR−/PR− and PR−/PR dimers. Thus, the observed reduction is PR activity is similar to what would be expected if true trans-dominant complementation was achieved during co-expression.

Co-Expressed Gag Precursor is not Processed by the GagPol PR in Trans.

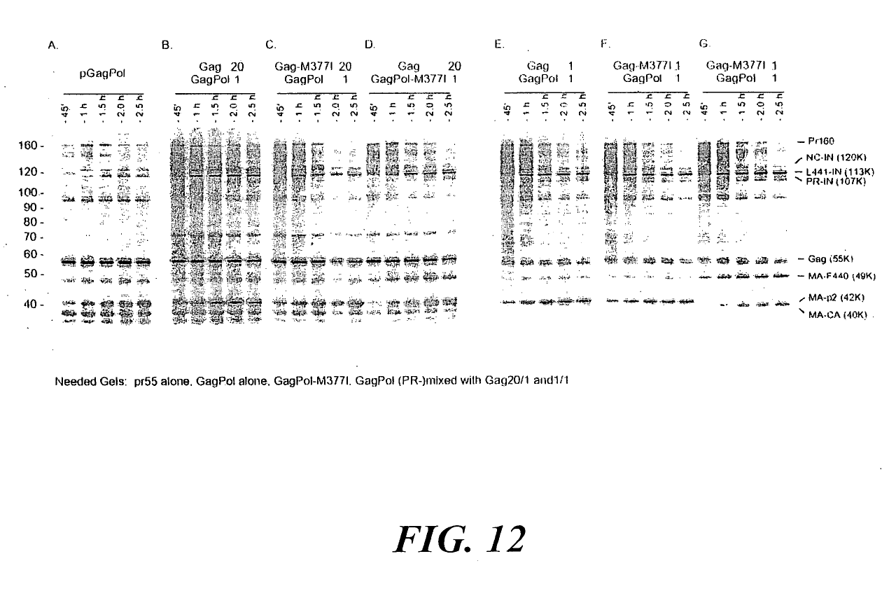

During viral assembly, two precursors are produced, Gag and GagPol. Gag is translated at a level approximately 20-fold greater than that of GagPol and encodes the structural proteins of the viral core. Because the Gag precursor terminates before the PR coding domain, during virus assembly, the Gag precursor must be cleaved by the PR in trans, either by the PR dimer embedded within the GagPol precursor or by the mature, fully processed PR dimer. To characterize the activity of the embedded PR further, the previous observations were extended to the processing of the Gag precursor (FIG. 12).

The two precursors were co-expressed at a 20:1 Gag to GagPol ratio in the RRL. Overall, the 55 kDa Gag precursor does not appear to undergo processing to any significant extent in these experiments. Co-expression of the wild type Gag construct together with GagPol did not influence GagPol processing (FIG. 12, Panel B). Similarly, there was no evidence that a Gag precursor containing an M377I substitution was processed in trans (FIG. 12, Panel C). Further, processing of a GagPol precursor containing an M377I substitution was unaffected by the presence of Gag (FIG. 12, Panel D). Therefore, despite the 20-fold excess of Gag precursor, no intermolecular cleavage is apparent in this system. As expected, similar results were obtained when the Gag and GagPol precursors were expressed at a ratio of 1:1 (FIG. 12, Panels E, F and G).

### EXAMPLE 5

### Materials and Methods—Study 3

The results above indicated that, in addition to the protease dimer interface, several other GagPol regions are important determinants in protease activity. Therefore, there are targets within at least the matrix, capsid, reverse transcriptase and integrase regions of the GagPol precursors that can be used to identify inhibitors of protease activation.

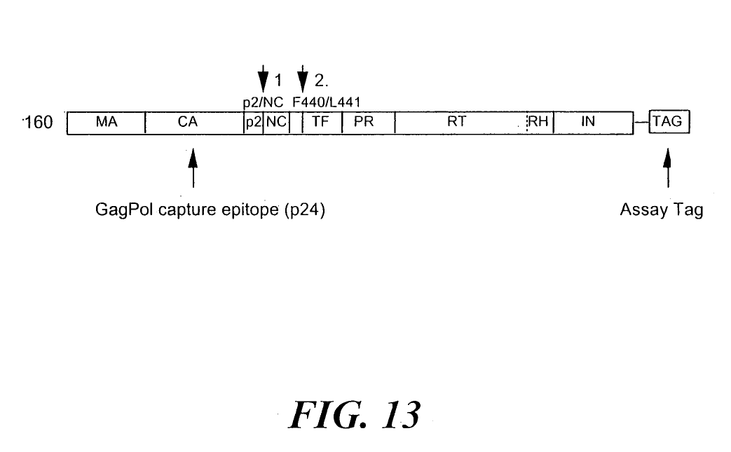

An assay was developed based on the studies of GagPol processing by the embedded protease in Examples 1-4 above. This assay takes advantage of the observation that when expressed in a RRL system, the activated protease cleaves the precursor twice, once at the junction of the p2 and nucleocapsid proteins and once within the transframe region (FIG. 13). Additional cleavages may be observed under different conditions. These two cleavages result in an approximately 41 kDa N-terminal fragment that contains the matrix (p17) and capsid (p24) proteins and a larger C-terminal fragment. Commercially available p24 ELISA plates (Coulter), in a 96-well format can be used to capture Gag and GagPol fragments that contain the HIV capsid p24 antigen recognized by the ELISA antibody.

FIG. 13 illustrates a rapid screen using protease activation as a screen for inhibitors that disrupt protease activity. Full-length GagPol (or a fragment containing protease and one or more of the indicated cleavage sites) is transcribed and translated in vitro in the presence of a potential inhibitor and captured on ELISA plates with an anti-p24 antibody directed against the HIV p24 capsid antigen. The GagPol precursor or fragment has a detectable Tag attached thereto. The captured GagPol or fragment is then assayed for the presence of the Tag. Examples of Tags include the influenza HA epitope (e.g., for an ELISA based assay) and luciferase (e.g., can be detected by chemical luminescence). If the protease is activated during synthesis of GagPol (or fragment) in the RRL, GagPol is cleaved and the C-terminal Tag is removed. If, however, the compound inhibits protease activation, the Tag remains linked to the plate through the p24 antibody. The assay is described further below.

Plasmids.

The plasmid pGPfs (see Example 1) was modified to add a detectable moiety to its C-terminus. A multiple cloning site polylinker was added to the C-terminus of pGPfs and two different detectable moieties were inserted into cloning sites in this polylinker, luciferase and an HA epitope tag (YPYDVPDYA; SEQ ID NO:4). Both constructs (pGPfs-luc and pGPfs-HA) underwent cleavage at rates similar to the wild type construct when expressed in a rabbit reticulocyte assay (RRL).

Protein Expression.

The pGPfs-HA construct was expressed in a RRL as described in Example 1. Briefly, coupled transcription and translation of pGPf-HA were carried out in RRL using the TNT system (Promega) in 200 μl reactions with 20 μCi of [³⁵S] cysteine (>1000 ci/mM Amersham Pharmacia Biotech) in the wells of the Coulter p24 ELISA plate (i.e., an antibody against the capsid p24 epitope is affixed to the surface of the plates). Reactions can be terminated either by adding LDS-polyacrylamide gel electrophoresis (LDS-PAGE) loading buffer (Invitrogen), by increasing the temperature to 90° C. or by increasing the pH to 9.0.

p24 ELISA.

The ELISA assays were conducted according to the instructions of the manufacturer (Coulter). 200 μl of the RRL sample was added to a well in the ELISA plate together with 20 μl 5% Triton X-100. The samples were allowed to incubate at 37° C. for one hour. Unbound protein was removed through a washing step (300 μl washes/well×6).

HA Detection.

The biotinylated HA monoclonal antibody was added to the ELISA plates and allowed to incubate with the bound GPfs for 1 hr at 37° C. The presence of the anti-HA monoclonal antibody was detected by the addition of streptavidin and read in a colometric format.

### EXAMPLE 6

### Results—Study 3

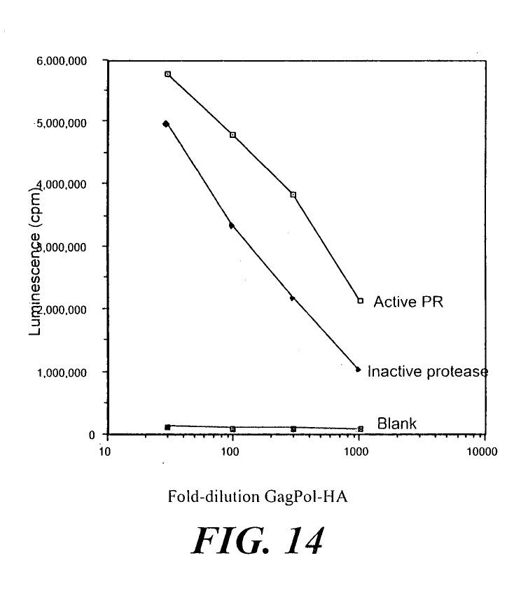

Two constructs encoding a full-length GagPol with a C-terminal influenza HA tag were transcribed and translated in a RRL in an anti-p24 ELISA plate as described above. One construct encodes an active protease, whereas the other encodes an inactive protease having an active site mutation (see above). FIG. 14 illustrates the detectability of GagPol-HA with active and inactive protease at varying dilutions of GagPol-HA on anti-p24 plates. GagPol-HA was translated and then diluted to the indicated dilution (concentration of the undiluted stock was 1 μM). The results indicate that maximum signal is given at low dilutions (high concentrations) of protein. The curve for the inactive protease corresponds to that which would be obtained in the presence of 100% inhibition of the PR by an inhibitor compound as compared with the curve for the fully active protease.

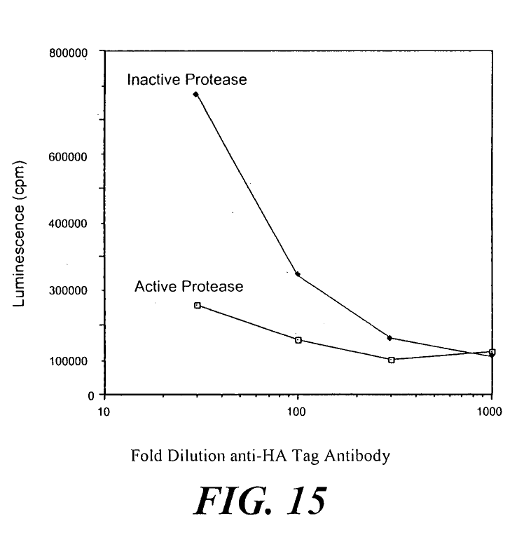

The effect of anti-HA Tag antibody dilution on the detection of GagPol-HA with active and inactive protease as captured on anti-p24 plates is shown in FIG. 15. Again, the curve for the inactive protease corresponds to the curve that would be observed in the presence of a completely inhibitory compound. At lower dilutions of the anti-HA antibody, the ability to detect the HA tag present is increased.

The foregoing is illustrative of the present invention, and is not to be construed as limiting thereof. The invention is defined by the following claims, with equivalents of the claims to be included therein.

1. A method of identifying an inhibitor of retrovirus protease activity, comprising:
(a) providing a nucleic acid that encodes a retrovirus GagPol or a fragment thereof comprising a protease, a protease cleavage site, a tether and a detectable moiety, wherein either the tether or the detectable moiety is located N-terminal to the cleavage site and the other is located C-terminal to the protease cleavage site;
(b) expressing the nucleic acid to produce the retrovirus GagPol or fragment thereof;
(c) binding the retrovirus GagPol or fragment thereof to a substrate comprising a binding partner for the tether such that the retrovirus GagPol or fragment thereof is bound via the tether to the substrate;
(d) contacting the retrovirus GagPol or fragment thereof with a candidate compound;
(e) removing released proteolytic products comprising the detectable moiety; and
(f) detecting the level of the detectable moiety bound to the substrate wherein persistence of the detectable moiety is indicative of an inhibitor of retrovirus protease activity.

2. The method according to claim 1, wherein the retrovirus is a Human Immunodeficiency Virus (HIV).

3. The method according to claim 1, wherein the retrovirus is a resistant retrovirus strain.

4. The method according to claim 1, wherein the nucleic acid encodes a retrovirus GagPol fragment comprising the retrovirus protease and transframe protein.

5. The method according to claim 4, wherein the fragment further comprises the retrovirus nucleocapsid protein.

6. The method according to claim 5, wherein the fragment further comprises the retrovirus p2 protein.

7. The method according to claim 6, wherein the fragment further comprises the retrovirus capsid protein.

8. The method according to claim 7, wherein the fragment further comprises the retrovirus matrix protein.

9. The method according to claim 1, wherein the nucleic acid encodes a retrovirus GagPol fragment comprising the retrovirus protease and the retrovirus reverse transcriptase.

10. The method according to claim 9, wherein the fragment further comprises the retrovirus integrase.

11. The method according to claim 1, wherein the nucleic acid encodes the retrovirus GagPol.

12. The method according to claim 1, wherein the tether is an epitope within the retrovirus GagPol or fragment thereof.

13. The method according to claim 7, wherein the tether is an epitope within the retrovirus capsid protein.

14. The method according to claim 1, wherein the binding partner for the tether is an antibody.

15. The method according to claim 1, wherein the detectable moiety is selected from the group consisting of luciferase, hemagglutinin antigen, maltose binding protein, c-myc, FLAG epitope, glutathione-S-transferase, fluorescent moiety, β-glucuronidase, alkaline phosphatase and β-galactosidase.

16. The method according to claim 1, wherein the detectable moiety is an epitope within the retrovirus GagPol or fragment thereof.

17. The method according to claim 1, wherein the method comprises an ELISA-based assay.

18-19. (canceled)

20. A kit for identifying inhibitors of retrovirus protease activity, comprising:
(a) a nucleic acid that encodes a retrovirus GagPol or a fragment thereof comprising a protease, a protease cleavage site, a tether and a detectable moiety, wherein either the tether or the detectable moiety is located N-terminal to the cleavage site and the other is located C-terminal to the protease cleavage site, such that cleavage at the protease cleavage site results in release of a proteolytic product comprising the detectable moiety; and
(b) a substrate comprising a binding partner for the tether.

21-35. (canceled)

36. A nucleic acid that encodes a retrovirus GagPol or a fragment thereof comprising a protease, a protease cleavage site, an exogenous tether and an exogenous detectable moiety, wherein either the tether or the detectable moiety is located N-terminal to the protease cleavage site and the other is located C-terminal to the protease cleavage site.

37. A vector comprising the nucleic acid of claim 36.
